(19) United States
(12) Patent Application Publication
(10) Pub. No.: US 2023/0284714 A1
(43) Pub. Date: Sep. 14, 2023

(54) ADAPTIVE PERSONAL PROTECTIVE FACIAL GARMENTS AND METHODS OF OPERATING THE SAME

(71) Applicant: MYANT INC., Toronto (CA)

(72) Inventors: Tony CHAHINE, Toronto (CA); Milad ALIZADEH-MEGHRAZI, Toronto (CA); Godfried Gysbrecht EDELMAN, Etobicoke (CA); Ladan ESKANDARIAN, Toronto (CA); John PERSIC, Toronto (CA); Sahar GOLMOHAMMADI ROSTAMI, Toronto (CA)

(21) Appl. No.: 18/018,110

(22) PCT Filed: Jul. 28, 2021
(86) PCT No.: PCT/CA2021/051062

§ 371 Date: Jan. 26, 2023

Related U.S. Application Data
(60) Provisional application No. 63/085,841, filed on Sep. 30, 2020.
(60) Provisional application No. 63/058,959, filed on Jul. 30, 2020.

(51) Int. Cl. A41D 13/11; A62B 23/02; A61B 5/00; A61B 5/024

(52) U.S. Cl. CPC A41D 13/1107; A62B 23/025; A61B 5/6803; A61B 5/02416; A41D 13/1192

(57) ABSTRACT

Embodiments of facial garments are disclosed. Garments may include textile components having conductive paths for interconnecting sensors, actuators, or other computing devices. Garments may be facial garments configured to capture user-related input via one or more sensor devices and provide output at an actuating structure in response to the captured user-related input. The facial garment may include textile components having silver or copper yarns knitted therein to provide an anti-microbial or anti-viral barrier between the user and the user's environment. A power source may provide power through the silver or copper fibers to provide a heating surface at a surface of the facial garment or to increase effectiveness of anti-microbial or anti-viral barrier provided by the textile.

### CROSS-REFERENCE TO RELATED APPLICATIONS

The present application claims priority from U.S. provisional patent application No. 63/085,841, entitled “ADAPTIVE PERSONAL PROTECTIVE FACIAL GARMENTS AND METHODS OF OPERATING THE SAME”, filed on Sep. 30, 2020, and from U.S. provisional patent application No. 63/058,959, entitled “ADAPTIVE PERSONAL PROTECTIVE FACIAL GARMENTS AND METHODS OF OPERATING THE SAME, filed on Jul. 30, 2020, the entire contents of which are hereby incorporated by reference herein.

### FIELD

The present disclosure generally relates to textile computing systems, and in particular to adaptive personal protective garments.

### BACKGROUND

Personal protective equipment may include articles of clothing or devices used to provide a physical barrier between a user and viral/bacterial specimens, dust, smoke, or other substances foreign to the user. Personal protective equipment may include medical gloves, gowns, aprons, face masks, face shields, hazmat suits, or other similar garments. In some examples, personal protective equipment may be one-time use garments. In some other examples, personal protective equipment may be sterilized or otherwise processed for multiple uses/re-uses.

### SUMMARY

Embodiments of the present disclosure describe garments including textile components having conducting paths for interconnecting sensor or actuator devices. In some embodiments, garments may be facial garments configured to capture user-related input via one or more sensor devices, such as temperature data, humidity data, bio-marker data, or the like, and provide an output at an actuating structure in response to the captured user-related input. In some embodiments, the facial garment may include textile components having silver or copper yarns knitted therein to provide an anti-microbial or anti-viral barrier between the user and the user's environment.

In some embodiments, the facial garment may be configured to couple to a power source. The power source may provide power through the silver or copper yarns to provide heating at a surface of the facial garment and to increase effectiveness of anti-microbial or anti-viral barrier provided by knitted copper or silver therein.

In one aspect, the present disclosure provides a facial garment. The facial garment may include: an attachment member for attaching the facial garment to a user; a textile mask body coupled to the attachment member, the textile mask body may include: a respiration region adapted to cover a portion of an oral-nasal region when the garment is attached to the user, the respiratory region including a sensory structure; a peripheral region adjacent the respiration region adapted to cover a portion of the user's cheek when the mask is worn by the user, the peripheral region including an electro-mechanical structure; and a conductive fiber network electrically coupling the respiration region and the peripheral region; and a computing device coupled via the conductive fiber network to the textile mask body, the computing device including a processor and a memory coupled to the processor, the memory storing processor executable instructions that, when executed, configure the processor to detect sensor data from the sensory structure and transmit actuating signals to the electro-mechanical structure.

In some embodiments, the conductive fiber network includes at least one interface for coupling to an add-on sensory device.

In any of the above embodiments, the sensory structure may include an environment sensor configured to detect at least one of pressure, temperature, humidity, carbon dioxide, carbon monoxide, volatile organic compounds, or gaseous air quality gases.

In any of the above embodiments, the electro-mechanical structure may include shape-shifting textile to provide increased fit or comfort.

In any of the above embodiments, the respiration region may include a nasal sub-region adapted to cover a nasal region of the user and an oral sub-region adapted to cover an oral region of the user, and the respiration region may include at least one cavity structure directing airflow among the nasal region and the oral region of the user.

In any of the above embodiments, the computing device may include at least one of an accelerometer, a gyroscope, or a magnetometer.

In any of the above embodiments, the facial garment may include a formed pocket layer positioned at the respiration region adapted to receive a filtration insert.

In some embodiments, the facial garment may include at least one of an N95 filter insert, a copper-treated nylon insert, BIOSA enzyme-contained film insert, or a non-woven sheet insert removably positioned in the formed pocket layer at the respiration region.

In any of the above embodiments, the textile mask body may include at least one of copper or silver yarn.

In any of the above embodiments, the textile mask body may include hydrophobic yarn fibers on an exterior portion of the textile mask body to repel or prevent virus or bacteria infected droplets from penetrating to the interior portion of the textile mask body.

In any of the above embodiments, the textile mask body may include a conductive yarn including at least one of silver or copper, and the conductive yarn may be configured to provide at least one of generated heat, increased anti-microbial or anti-viral properties with increasing temperature, or a temperature sensor.

In any of the above embodiments, the textile mask body may include a conductive yarn arranged with insulative yarns to provide an electrostatic charge to provide antimicrobial or anti-viral properties.

In any of the above embodiments, the textile mask body may include at least one sensor configured to detect oximetry.

In some embodiments, the at least one sensor may include a photoplethysmogram (PPG) sensor for sensing when oxygen level of the user is decreasing.

In any of the above embodiments, the textile mask body may include a form fitting yarn configured to heat shrink to provide form fit to the user's face.

In any of the above embodiments, the textile mask body may include shape memory yarn to provide a form fit to the user's face to reduce air gaps between the textile mask body and the user's face.

In any of the above embodiments, the textile mask body may include an infrared sensor positioned proximal to an ear of the user.

In any of the above embodiments, the textile mask body may include a photoplethysmogram (PPG) sensor configured to detect heart rate monitoring statistics.

In various further aspects, the disclosure may provide corresponding systems and devices, and logic structures such as machine-executable coded instruction sets for implementing such systems, devices, and methods.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many further features and combinations thereof concerning embodiments described herein will appear to those skilled in the art following a reading of the present disclosure.

### DESCRIPTION OF THE FIGURES

In the figures, embodiments are illustrated by way of example. It is to be expressly understood that the description and figures are only for the purpose of illustration and as an aid to understanding.

Embodiments will now be described, by way of example only, with reference to the attached figures, wherein in the figures:

FIG. 1 illustrate a perspective view of a mask, in accordance with an embodiment of the present disclosure;

FIG. 2 illustrates a rear perspective view of the mask of FIG. 1;

FIGS. 3A and 3B illustrate an inner textile layer and a filtration insert, in accordance with an embodiment of the present disclosure;

FIG. 4 illustrates a perspective view of a mask, in accordance with an embodiment of the present disclosure;

FIG. 5 illustrates a front-elevation view of the mask of FIG. 5;

FIG. 6 illustrates a side-elevation view of the mask of FIG. 4;

FIG. 7 illustrates an exploded view of the mask of FIG. 4;

FIG. 8 illustrates a rear perspective view of the mask of FIG. 4;

FIG. 9 illustrates a cut-away, rear-perspective view of the mask illustrated in FIG. 8;

FIG. 10 illustrates a rear perspective view of a mask, in accordance with embodiments of the present disclosure;

FIG. 11 illustrates an exploded view and a partially exploded view of a mask, in accordance with an embodiment of the present disclosure;

FIG. 12 illustrates an enlarged, cutaway view of a shape-shifting filter illustrated in FIG. 11;

FIG. 13 illustrates an enlarged, partial cutaway view of the shape-shifting filter of FIG. 11;

FIG. 14 illustrates a perspective view of the shape-shifting filter of FIG. 11;

FIG. 15A illustrates a side, cross-sectional view of a textile mask body positioned about an oral-nasal cavity region of a user, in accordance with an embodiment of the present disclosure;

FIG. 15B illustrates a rear perspective view of a facial garment, in accordance with an embodiment of the present disclosure;

FIG. 16 illustrates a perspective view of a facial garment, in accordance with an embodiment of the present disclosure;

FIG. 17 illustrates a perspective view of a facial garment, in accordance with an embodiment of the present disclosure;

FIG. 18 illustrates a perspective view of a facial garment, in accordance with an embodiment of the present disclosure;

FIG. 19 illustrates a rear plan view of the facial garment of FIG. 15;

FIG. 20 illustrates a top view of a sensor or actuator knitted or integrated in a textile, in accordance with an embodiment of the present disclosure;

FIG. 21A illustrates a side elevation view of a sensor or actuator knitted or integrated in a textile, in accordance with an embodiment of the present disclosure;

FIG. 21B illustrates a perspective view of a sensor or actuator knitted or integrated into a textile, in accordance with an embodiment of the present disclosure;

FIG. 22 illustrates a rear perspective view of a facial garment, in accordance with an embodiment of the present disclosure;

FIG. 23 illustrates a rear perspective view of a facial garment, in accordance with an embodiment of the present disclosure;

FIG. 24 illustrates a block diagram of a computing device, in accordance with an embodiment of the present disclosure;

FIG. 25 illustrates an exploded, rear perspective view of a mask, in accordance with an embodiment of the present disclosure;

FIG. 26 illustrates an exploded, front perspective view of the mask of FIG. 25;

FIG. 27 illustrates a front perspective view of the mask 2500 of FIG. 25 fitted to a user's head;

FIG. 28 illustrates a front elevation view of the mask of FIG. 25 fitted to a user's head;

FIG. 29 illustrates a right side elevation view of the mask of FIG. 25 fitted to a user's head;

FIG. 30 illustrates a left side elevation view of the mask of FIG. 25 fitted to a user's head; and

FIG. 31 illustrates a front perspective view of a mask, in accordance with an embodiment of the present disclosure.

### DETAILED DESCRIPTION

The term “connected” or “coupled to” may include both direct coupling (in which two elements that are coupled to each other contact each other) and indirect coupling (in which at least one additional element is located between the two elements).

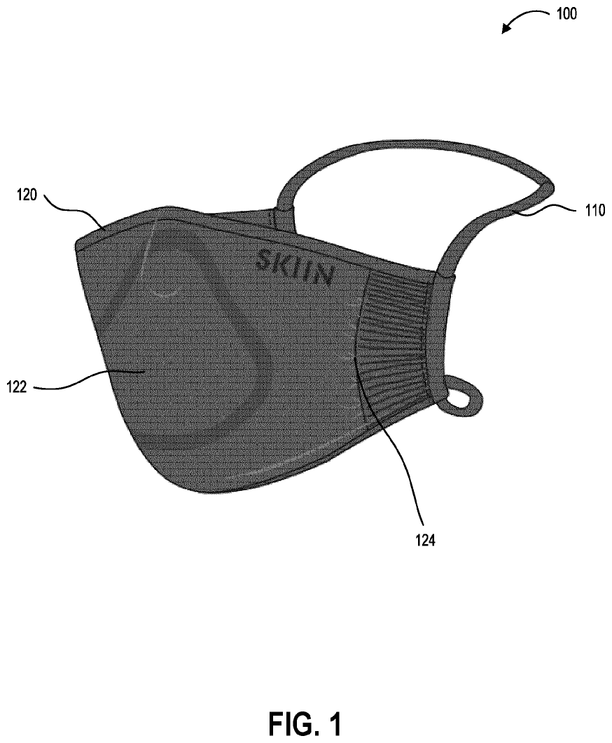

Reference is made to FIG. 1, which illustrates a perspective view of a mask 100, in accordance with an embodiment of the present disclosure. The mask 100 may be adapted to be worn over the mouth or the nose of a user to protect the user's respiratory system. In some examples, the mask 100 may be configured to filter out undesirable substances such as dust, smoke, biological material, or other gaseous, liquid, or solid materials. In such examples, mask 100 may function as a respirator device. In some examples, the mask 100 may be configured to sense particular substances. As a physical barrier, in some situations, the mask 100 may prevent spread to the environment of gases, liquids, or solids that may be expelled from the user's respiratory system.

The mask 100 may include an attachment member 110. The attachment member 110 is configured to securely attach the mask 100 to the user. In some embodiments, the attachment member 110 may include one or more loops configured to wrap around the head or ears of the user. In some embodiments, the attachment member 110 may include elastic material or adjustment features allowing the attachment member 110 to be tightened or loosened to adapt to physical features of the user. One or more different geometric configurations of the attachment member 110 may be contemplated.

The mask 100 may include a textile mask body 120 coupled to the attachment member 110. The textile mask body 120 may include a textile material consisting a network of natural or synthetic fibers, such as animal-based material (e.g., wool or silk), plant-based material (e.g., linen or cotton), or synthetic material (e.g., polyester or nylon). Other types of textiles may be contemplated.

The textile mask body 120 may include electrical, mechanical, or electro-mechanical textile structure integrated therein. In some embodiments, the textile mask body 120 may include a conductive fiber network coupling two or more portions of the textile mask body 120. For example, the conductive fiber network may include power or data transmissions structures. The conductive fiber network may be configured as a bi-directional bus for transmitting or receiving signals, such as data signals, power signals, or other type of signals that may be carried on the conductive fiber network.

In some embodiments, the textile mask body 120 may include electrical conductive circuits, sensors, actuators, or other types of data acquisition or feedback components. In some examples, electrical, mechanical, or electro-mechanical fibers, such as piezoelectric, electromagnetic, shape shifting, etc. yarns, may be knitted or weaved into a textile fabric. For instance, electro-mechanical fibers may be knitted or weaved in a “zig-zag” pattern across textile fabric to provide sensor or actuator structures. Conductive paths or structures may be integrated into textiles by one or a combination of methods including inlaying, knitting, weaving, embroidery, adhesive bonding, or mechanical bonding. Other methods of integrating conductive paths into textile structures may be contemplated.

In the illustrated embodiment, textile mask body 120 is formed of a knitted textile. Textile mask body 120 includes a plurality of conductive fibres interlaced with a plurality of non-conductive fibres. The conductive fibres define a plurality of signal paths suitable for delivering data and/or power, e.g., to form a conductive fiber network.

In some embodiments, textile mask body 120 may be formed of other textile forms and/or techniques such as weaving, knitting (warp, weft, etc.) or the like. In some embodiments, textile mask body 120 includes any one of a knitted textile, a woven textile, a cut and sewn textile, a knitted fabric, a non-knitted fabric, in any combination and/or permutation thereof. Example structures and interlacing techniques of textiles formed by knitting and weaving are disclosed in U.S. Patent Application No. U.S. Ser. No. 15/267,818, entitled “Conductive Knit Patch”, the entire contents of which are herein incorporated by reference.

As used herein, “textile” refers to any material made or formed by manipulating natural or artificial fibres to interlace to create an organized network of fibres. Textiles may be formed using yarn, where yarn refers to a long continuous length of a plurality of fibres that have been interlocked (i.e. fitting into each other, as if twined together, or twisted together). Herein, the terms fibre and yarn may be used interchangeably. Fibres or yarns can be manipulated to form a textile according to any method that provides an interlaced organized network of fibres, including but not limited to weaving, knitting, sew and cut, crocheting, knotting and felting.

Different sections of a textile can be integrally formed into a layer to utilize different structural properties of different types of fibres. For example, conductive fibres can be manipulated to form networks of conductive fibres and non-conductive fibres can be manipulated to form networks of non-conductive fibers. These networks of fibres can comprise different sections of a textile by integrating the networks of fibres into a layer of the textile. The networks of conductive fibres can form one or more conductive pathways that electrically connect with actuators and sensors embedded in textile mask body 120, for conveying data and/or power to and/or from these components.

In some embodiments, multiple layers of textile can also be stacked upon each other to provide a multi-layer textile.

As used herein, “interlace” refers to fibres (either artificial or natural) crossing over and/or under one another in an organized fashion, typically alternately over and under one another, in a layer. When interlaced, adjacent fibres touch each other at intersection points (e.g. points where one fibre crosses over or under another fibre). In one example, first fibres extending in a first direction can be interlaced with second fibres extending laterally or transverse to the fibres extending in the first connection. In another example, the second fibres can extend laterally at 90° from the first fibres when interlaced with the first fibres. Interlaced fibres extending in a sheet can be referred to as a network of fibres.

As used herein “integrated” or “integrally” may refer to combining, coordinating or otherwise bringing together separate elements so as to provide a harmonious, consistent, interrelated whole. In the context of a textile, a textile can have various sections comprising networks of fibres with different structural properties. For example, a textile can have a section comprising a network of conductive fibres and a section comprising a network of non-conductive fibres. Two or more sections comprising networks of fibres are said to be “integrated” together into a textile (or “integrally formed”) when at least one fibre of one network is interlaced with at least one fibre of the other network such that the two networks form a layer of the textile. Further, when integrated, two sections of a textile can also be described as being substantially inseparable from the textile. Here, “substantially inseparable” refers to the notion that separation of the sections of the textile from each other results in disassembly or destruction of the textile itself.

In some examples, conductive fabric (e.g. group of conductive fibres) can be knit along with (e.g. to be integral with) the base fabric (e.g. surface) in a layer. Such knitting may be performed using a circular knit machine or a flat bed knit machine, warp knit, or the like, from a vendor such as Santoni, Stoll, or Karl Mayer.

The textile mask body 120 may include conductive yarns such as silver coated yarns or copper micro wire covered (wrapped) or twisted on an insulated yarn. The conductive yarn may include at least one of the following beneficial features: (1) the conductive yarn may act as a heating element with electrical current running through it, (2) the conductive yarn may act as a temperature sensor using a length of conductive yarn which varies its resistance with temperature, or (3) the conductive yarn may include be configured to provide an improved anti-microbial or anti-viral barrier between a user of the textile mask body 120 and the user's environment. Conductive yarns may be surrounded by highly insulating yarns to setup an electrostatic field for providing anti-viral properties. Heating the incoming air may provide advantages in cold climates and with the added benefit of preventing cold air into the lungs, which may have benefits to asthmatics and individuals with breathing issues. Temperature measurements may be carried out with the measurement of electrical resistance changes over a given length of conductive yarn over a temperature change. Higher temperatures and humidity may improve the efficacy of silver and copper as shown in an article: H. Michels, J. Noyce, and C. Keevil, “Effects of temperature and humidity on the efficacy of methicillin resistant Staphylococcus aureus challenged antimicrobial materials containing silver and copper,” Lett. Appl. Microbiol., vol. 49, no. 2, pp. 191-195, August 2009, doi: 10.1111/j.1472-765X.2009.02637.x.

The textile mask body 120 may include a respiration region 122 including one or more sensory structures adapted to cover a portion of an oral-nasal region of a user when the mask is worn by the user. In some embodiments, the respiration region 122 may include a nasal sub-region adapted to cover a nose of the user and an oral sub-region adapted to cover a mouth of the user.

The textile mask body 120 may include a peripheral region 124 including one or more electro-mechanical structures adjacent the respiration region. The peripheral region 124 may be adapted to cover a portion of a user cheek when the mask is worn by the user. In some embodiments, the peripheral region 124 may include textile features adapted to minimize gaps or open spaces between the cheek of the user and the textile mask body 120. For example, at least a portion of the peripheral region 124 may include textile portions having elastic strands that may stretch or retract for adapting to the contour of the user's face.

In some situations, the user may wear the mask 100 for relatively long durations of time. As the mask 100 may be a physical barrier positioned between the user's face and the environment, in some situations, the mask 100 may impede air flow to the user's face and the user may be breathing in air that may have low oxygen content or may be breathing in substantially the same air that the user has breathed out. It may be beneficial to provide mask structures to increase air flow to the nasal-oral region of the user's face while the mask 100 may be worn.

In some embodiments, the textile mask body 120 may include spacer structures to provide increased air circulation. For example, the mask body 120 may include a spacer fabric and shape-shifting materials to provide thermal regulation and moisture management properties. The orientation of spacer fabric or shape-shifting materials may configure functionality of the textile mask body 120 for thermoregulation, breathability, or moisture management. The textile mask body 120 may include an embedded spacer fabric. In some examples, the outer layers of this spacer fabric may be constructed differently. Material types and the surface characteristics of the layers may influence the elastic and comfort properties of the whole structure and the moisture transport and air circulation level between the layers. Because of the latter two functionalities, heat congestion and maceration of the user's skin may be reduced.

In some embodiments, knitted spacer fabrics may be provided by knitting techniques which promote adjustments to absorbency and water vapour permeability level of the textile mask body 120. Structural changes may easily and cost-effectively be set by controlling spacer yarn connecting distance, determining the number of elastic yarns to be used, or selecting the suitable type of spacer yarns and the process parameters. In some embodiments, the textile mask body 120 may include shapeshifting materials to control these specifications further. Shape shifting materials may be in the form of alloys or polymers. The alloys may be wrapped around other yarns or used as wires. Using shape memory alloys in the structure of the spacer fabric may assist with controlling the thickness of the spacer layer or with creating higher air circulation as needed.

In some embodiments, shape memory polymers may be used in different forms such as fibers, coatings, knitted or woven fabrics, and membranes. Shape memory polymers may be subject to or exhibit Micro-Brownian motion (thermal vibration) occurring within the materials when the temperature rises above a predetermined activation point. As a result of Micro-Brownian motion, molecules form free spaces (micro pores) in the material (fabric, membrane, etc.), thereby promoting water vapour and heat to escape or pass through. Because permeability increases as the temperature rises, the fabric/membrane may exhibit transitions in response to changes in the mask user's environment. In some situations, water vapour inside the structure (or on an interior surface of the textile mask body 120) may be absorbed before it has a chance to condense. The absorbed water vapour may be conducted into and diffused throughout the fabric/membrane/fiber and may then be emitted from the surface of the membrane/fiber/fabric.

In some scenarios, textiles having electrostatic charge thereon or textiles generating an electric field may be beneficial for acting as a barrier to virus or bacteria, or for eradicating virus or bacteria. In some embodiments, the textile mask body 120, or regions of the textile mask body 120, may include textile portions having an electrostatic charge thereon or textile portions for generating an electric field, thereby providing an anti-bacterial or anti-viral layer thereon.

In some embodiments, the textile mask body 120 may include at least one of silver or copper strands. For example, when two metals may become wet/humid (akin to a battery cell), the textile strands may trap or be attracted to relatively small particles having an opposite polarity. According, in some embodiments of the present disclosure, silver or copper strands may be knitted into the textile mask body 120. The metal strands may be suitable for or configured for providing electrostatic charge on an interior surface of the mask. In another example, the metal strands may be knitted to provide one or more poles on an inner layer of the textile mask body 120 and configured to receive electric current therein for trapping smaller particles of opposite polarity. In some embodiments, silver or copper strands may be knitted into the textile mask body 120 in one or more configurations (e.g., alternating rows of copper and silver strands, or other patterns).

To act as an effective physical barrier between the user and the environment, the mask 100 may need to be fitted to the user. It may be challenging to design a one-size-fits all mask for users having a range of dimensions and for users having a variety of face structures or contours. In some embodiments, the mask 100 may be configured for users having a targeted face size (e.g., large, medium, small, etc.) or face structure (e.g., round face, slender face, etc.). When the mask 100 may be worn for extended periods of time, body heat from the user or respiration from the user may heat up the mask. In some situations, it may be beneficial to provide mask structures for adaptively adjusting mask fit to the user to maintain the effective physical barrier between the user's face and the environment.

Further, masks may be constructed of textile materials that may impede respiration. The degree that respiration may be impeded by the mask may depend in part on the density of the textile material or how tightly knitted the textile or fabric of the mask may be. A mask constructed with a denser textile fabric may be relatively more effective at providing a barrier to substances foreign to the user but may reduce the user's ability to breathe. A mask constructed with lower density textile fabric may increase the user's ability to breath but may be less effective as a physical barrier to substance foreign to the user. It may be beneficial to provide mask devices constructed with textile materials for acting as an effective physical barrier to bacteria, viruses, or other foreign substances while maintaining relatively good breathability thereby allowing a user to wear masks for extended durations of time.

In some situations, deducing or identifying physiological data associated with a user may require invasive or intentional data collection using specialized medical equipment. In some situations, it may be beneficial to provide devices for deducing or identifying user bio-markers based on involuntary user activity, such as respiration. As masks may be adapted to be worn over a user's nose or mouth, it may be beneficial to provide mask structures for providing near-real-time or real-time physiological data and feedback to a user based on bio-markers associated with the user's respiratory system.

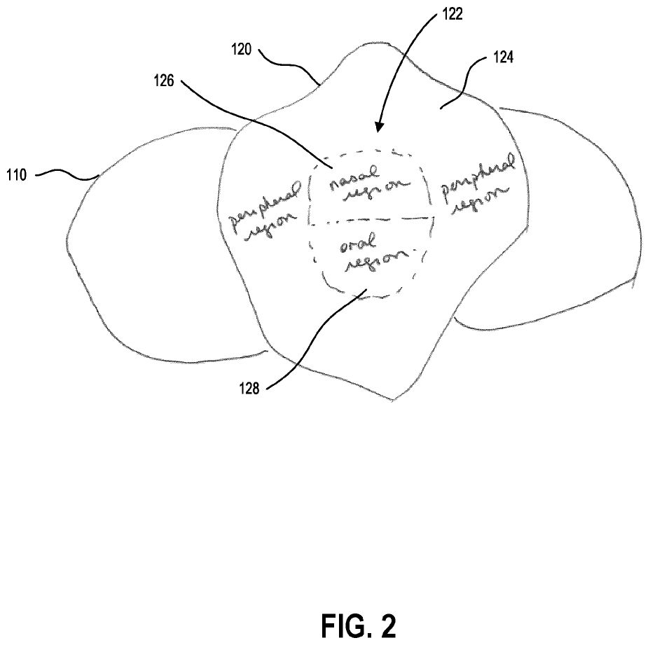

Reference is made to FIG. 2, which illustrates a rear perspective view of the mask 100 of FIG. 1. The respiration region 122 may be adapted to cover the oral-nasal region of the user when the mask is worn by the user. Further, the peripheral region 124 may be positioned adjacent the respiration region 122. When the mask 100 is worn by the user, the peripheral region 124 may be adapted to cover or interface with a portion of a user's cheek. The peripheral region 124 is shown in FIG. 2 as circumferentially surrounding the respiration region 122; however, it will be understood that the peripheral region 124 may be defined as localized regions that may be oriented based on other geometric configurations.

In some embodiments, the respiration region 122 may be divided into one or more sub-regions. For example, the respiration region 122 may include a nasal sub-region 126 adapted to cover a nasal region of the user. In particular, the nasal sub-region 126 may be geometrically adapted to cover the nose or nostrils of the user's nose when the mask 100 is worn by the user. One or more sensory structures may be positioned at the nasal sub-region 126 to detect bio-markers from respiratory output via the user's nostrils or detect other data based on respiratory input via the user's nostrils. In some embodiments, the nasal sub-region 126 may include one or more actuator structures configured to alter, in response to sensory input, physical features of the textile mask body 120.

In some embodiments, the respiration region 122 may include an oral sub-region 128 geometrically adapted to cover the mouth of the user when the mask 100 is worn by the user. The oral sub-region 128 may include one or more sensory structures configured to detect bio-markers from respiratory output via the user's mouth. In some embodiments, the oral sub-region 128 may include one or more actuator structures configured to alter, in response to sensory input, physical features of the textile mask body 120.

The peripheral region 124 may include electro-mechanical structures configured to alter, in response to sensory input, physical features of the textile mask body 120. In some embodiments, the peripheral region 124 may include shape-shifting yarns or structures that may be geometrically altered in response to sensory data.

In some embodiments, the peripheral region 124 may be constructed of shape-shifting yarns configured to change in shape in response to electrical current. Such shape-shifting yarns may be coupled to a computing device and/or a power source, and the computing device may include a processor and a memory storing processor-executable instructions that configure the processor to provide electrical current to the shape-shifting yarns to alter physical properties of the shape-shifting yarn. The processor may be configured to provide electrical current to the shape-shifting yarns in response to sensory data detected by sensory structures.

In some embodiments, the one or more sensory structures or actuator structures may be integrated in the textile mask body 120. For example, the structures may be integrated into the textile mask body 120 by methods including inlaying, knitting, weaving, adhesive bonding, or mechanical bonding. By integrating sensory structures or actuator structures into the textile mask body 120 during manufacturing, the manufacturing costs of the mask 100 may be reduced (as compared to an after-the-fact retrofit of sensory and actuator structures onto known masks). Further, in some configurations, integrating structures into the textile mask body 120 may result in slimmer form factors.

In some embodiments, the one or more sensory structures or actuator structures may be discrete structures that may be coupled to the textile mask body 120. For example, the textile mask body 120 may include a conductive fiber network having one or more socket interfaces for coupling a discrete sensory device or discrete actuator device to the textile mask body 120. In embodiments of the textile mask body 120 having one or more socket interfaces, the mask 100 may be configurable with sensor devices and/or actuator devices desirable for particular usage scenarios. For example, a mask configured for monitoring hydration status of a hospital patient may be configured with a different combination of sensory devices and/or actuator devices than a mask configured for a heath care professional wearing the mask over a 12 hour duration. Embodiments of combinations of sensory devices and/or actuator devices for masks may be contemplated in the present disclosure.

In some embodiments, the textile mask body 120 may include at least one of temperature sensors, humidity sensors, or breath sensors integrated thereon, and may include other sensory devices coupled thereon as off-the-shelf devices. Example off-the-shelf sensor devices that may be coupled to the textile mask body 120 include volatile organic compound (VOC) sensors (e.g., by Bosch). In some embodiments, an off-the-shelf sensor device that may be coupled to the textile mask body 120 may include sensors for detecting bio-markers, or the like, based on interactions with a user of the textile mask body 120.

In some embodiments, the peripheral region 124 may be constructed of shape-shifting yarns adapted to detect temperature changes and configured to alter shape or alter configuration in response to temperature changes. In some embodiments, the peripheral region 124 may include fibers having varying hydrophilic or hydrophobic properties, or fibers having varying thermal conductivities, thereby providing thermal regulation or moisture management features.

In some embodiments, the conductive fiber network may be integrated across numerous regions of the textile mask body 120 to couple the plurality of regions of the mask 100. For example, the conductive fiber network may couple features of the respiration region 122 to the peripheral region 124. In some examples, the conductive fiber network may couple features of the nasal sub-region 126 to the oral sub-region 128. Further, in some examples, the conductive fiber network may couple any one or more of the regions of the textile mask body 120 to a computing device.

In some embodiments, the computing device may be affixed to the attachment member and, when the mask is worn by the user, the computing device may be positioned behind the user's ear (e.g., akin to placement/positioning of a hearing aid).

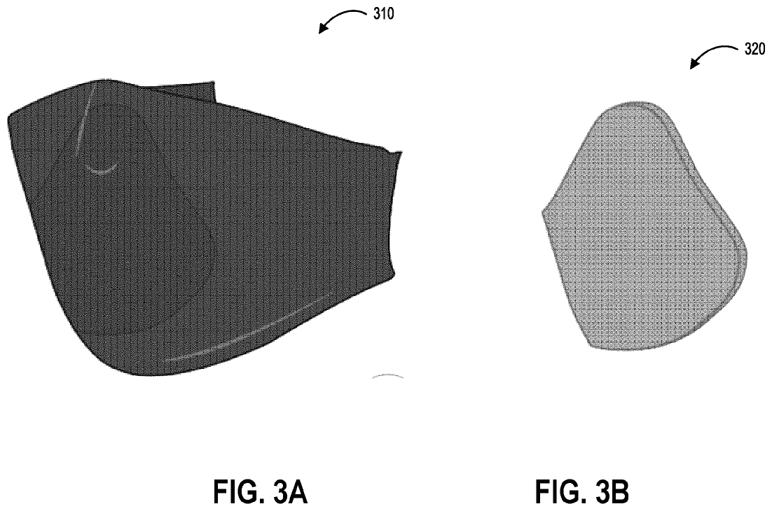

In some embodiments, the textile mask body 120 may be composed of a plurality of layers. The textile mask body 120 may include at least an outer textile layer, one or more pocket forming layers, and an inner textile layer. To illustrate features of one or more pocket forming layers or inner textile layers, reference is made to FIGS. 3A and 3B, which illustrate an inner textile layer 310 and a filtration insert 320, in accordance with an embodiment of the present disclosure.

In some embodiments, an outer textile layer may include a hydrophobic textile surface configured to wick moisture in a direction from the inner textile layer to the outer textile layer. Hydrophobic textile fibers may be intertwined with the outer textile layer to repel liquid droplets having undesirable micro-organisms, such as bacteria, viruses, or the like. In some embodiments, the outer textile layer may be infused or intertwined with copper or silver.

In some embodiments, the inner textile layer 310 may include hydrophilic properties configured to wick moisture away from the nasal-oral region of the user. In some embodiments, the inner textile layer 310 may be infused or intertwined with copper or silver. In some examples, the textile mask body 120 may include infused or intertwined with copper or silver at the respiratory region 122, thereby enhancing antibacterial or antiviral properties in areas having high moisture and/or high temperature based on respiration of the user.

In some embodiments, the textile mask body 120 may include one or more pocket forming layers. In some embodiments, the one or more pocket forming layers may be positioned at the respiratory region 122 of the textile mask body 120. In some embodiments, the one or more pocket forming layers may be a combination of the outer textile layer and the inner textile layer 310.

In some embodiments, the one or more pocket forming layers may also be positioned at one or more portions of the peripheral region 124 of the textile mask body 120. The pocket forming layers may be configured to receive the filtration insert 320 to supplement filtration or physical barrier properties of the textile mask body 120. In some embodiments, the filtration insert may be an N95 filter insert, a copper-treated nylon insert, a BIOSA enzyme-contained film inert, or a non-woven sheet insert that may be removably positioned in a formed pocket layer.

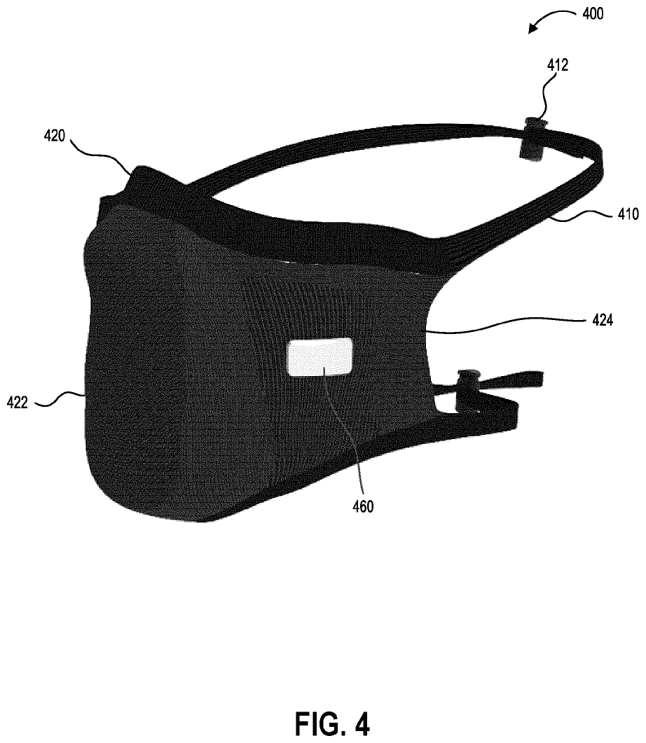

Reference is made to FIG. 4, which illustrates a perspective view of a mask 400, in accordance with an embodiment of the present disclosure. The mask 400 may be adapted to be worn over the mouth or the nose of a user to protect the user's respiratory system.

The mask 400 includes an attachment member 410. The attachment member 410 is configured to securely attach the mask 400 to the user. In some embodiments, the attachment member 410 may include one or more spring-loaded clamps 412 that may fasten ends of the attachment member 410. In some situations, the length or other dimension relative to a mask body 420 may be adjustable based on positioning of the one or more spring-loaded clamps 412 along the attachment member 410. Spring-loaded clamps 412 are shown in FIG. 4; however, in some other embodiments, other features for adjusting the attachment member 410 relative to the mask body 420 may be contemplated. Features for adjusting the attachment member 410 relative to the mask body 420 may allow the user to configure the mask 400 such that the mask 400 may be fitted to dimensions or shape of the user's head.

In some embodiments, the attachment member 410 may be configured to wrap around a head of the user such that the mask 400 may be secured to the user when worn over the mouth or nose of the user.

The mask body 420 may include textile material consisting of a network of natural or synthetic fibers, similar to the mask body 120 described with reference to FIG. 1. The mask body 420 may include electrical, mechanical, or electro-mechanical textile structures integrated therein. The mask body 420 may include a respiration region 422 adapted to cover a portion of an oral-nasal region of a user when the mask is worn by the user. Further, the mask body 420 includes a peripheral region 424 and may include one or more electrical, mechanical, or electro-mechanical structures adjacent the respiration region 422.

In some embodiments, the mask body 420 may include a computing device 460 coupled thereto. The computing device 460 may be coupled, via the textile material of the mask body 420, to electrical, mechanical, or electro-mechanical textile structures integrated in the mask body 420. In some embodiments, the textile material may be a conductive fiber network electrically coupling the computing device 460 to the respiration region 422 and/or the peripheral region 424.

In FIG. 4, the computing device 460 is positioned on the mask body 420; however, it may be contemplated that the computing device 460 may be affixed to the attachment member 410. When the mask 400 is worn by the user, the computing device may be positioned adjacent the user's cheek/face, positioned behind the user's ear (e.g., akin to placement/positioning of a hearing aid), or positioned behind the user's head (e.g., proximal to the one or more spring-loaded clamps 412).

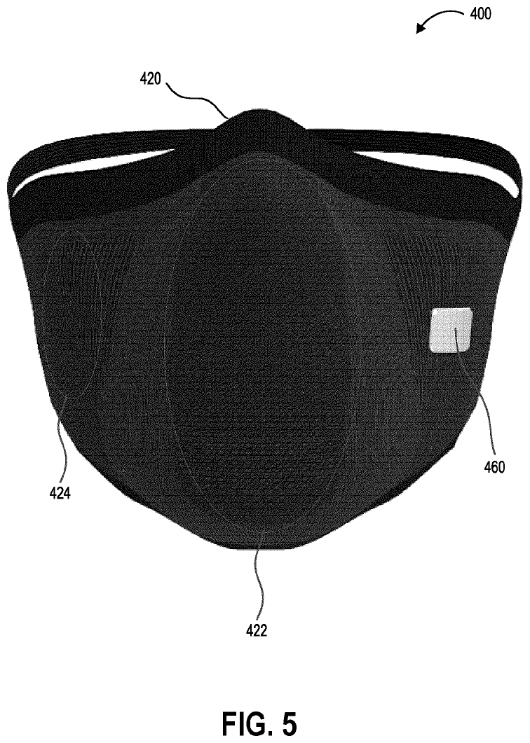

Reference is made to FIG. 5, which illustrates a front-elevation view of the mask 400 of FIG. 4. In FIG. 5, the respiration region 422 is illustrated as being positioned in a relatively central region of the mask body 420. In some embodiments, the peripheral region 424 is positioned at side regions or adjacent to the respiration region 422 of the mask body 420. In FIG. 4, one peripheral region 424 is highlighted; however, other areas adjacent the respiration region 422 may also include features of the peripheral region 424.

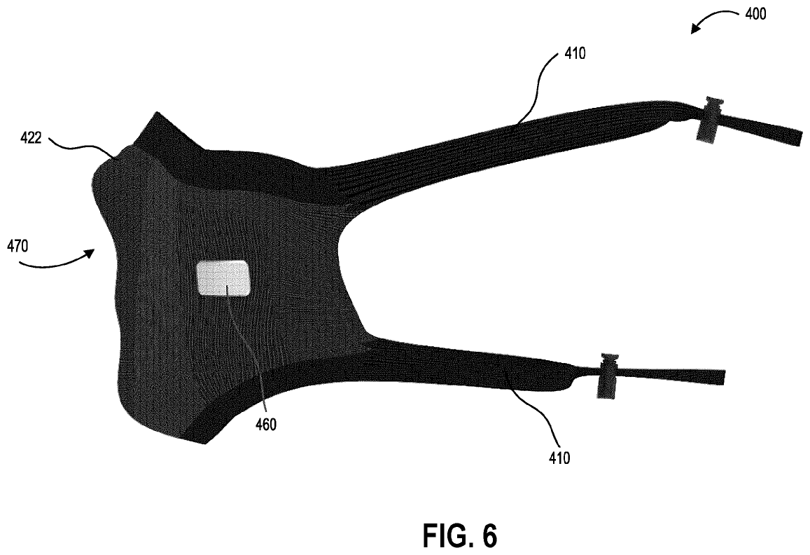

Reference is made to FIG. 6, which illustrates a side-elevation view of the mask 400 of FIG. 4. The attachment member 410 may include a pair attachment member portions adapted to wrap around a head of a user. A first of the pair of attachment member portions may wrap around an upper portion of the head of the user and a second of the pair of attachment member portions may wrap around a lower portion of the head when the mask 400 is secured to the user.

In some embodiments, the respiration region 422 may include a contoured textile portion adapted to follow the contour of the nasal-oral region of the user's face. The respiration region 422 may include a malleable contoured textile 470 adapted to retain a physical shape. Thus, in some situations, the user may configure the respiration region 422 such that the respiration region 422 may not touch the user's nose or lips when the mask 400 is worn by the user. When the respiration region 422 is contoured to not touch the user's nose or lips, there may be a void between the respiration region 422 of the mask body 420 and the user's nose or lips, allowing gases or liquids to flow within the void.

In some situations, the respiration region 422 may be configured to not directly touch the user's lips or nose to provide the user with greater comfort. In some situations, the respiration region 422 may be configured to not directly touch the user's lips or nose to reduce the chance of foreign liquids seeping through the mask body 420 immediately interacting with the user's nasal-oral cavity.

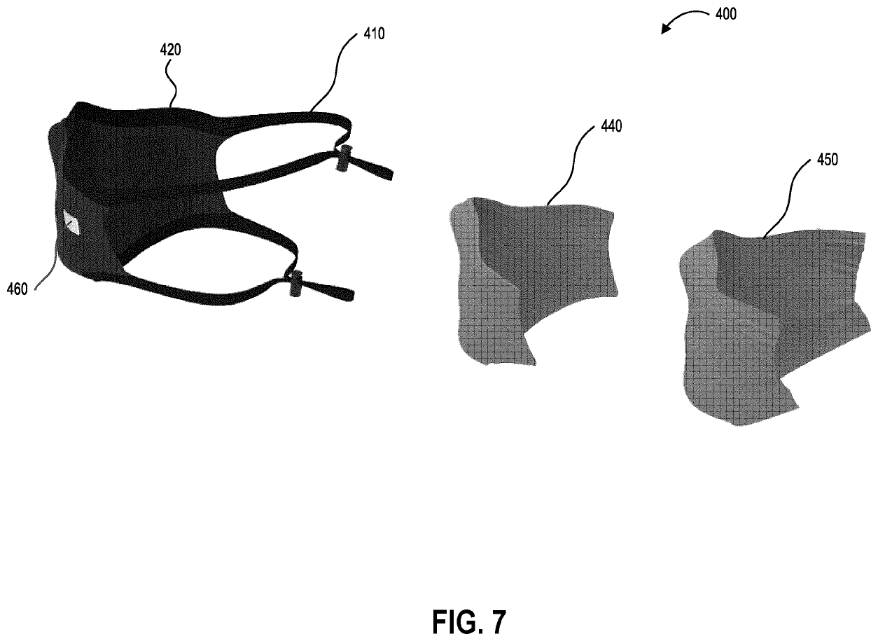

Reference is made to FIG. 7, which illustrates an exploded view of the mask 400 of FIG. 4. As described with reference to FIG. 4, the mask 400 includes one or more attachment members 410 coupled to the mask body 420. The computing device 460 may be coupled to a conductive fiber network of the textile mask body 420. In FIG. 7, the computing device 460 is illustrated as being positioned on the mask body 420.

The mask 400 may include a filtration insert 440 adapted to be placed between the mask body 420 and an inner textile layer 450. In some embodiments, the inner textile layer 450 may include a conductive fiber network adapted to electrically couple to the mask body 420 when the inner textile layer 450 is installed within the mask body 420.

In some embodiments, the inner textile layer 450 may include one or more sensory structures or actuator structures configured to deduce, detect, or identify user bio-markers based on involuntary user activity (e.g., respiration) when the mask 400 is worn by the user. As described in the present disclosure, the computing device may deduce or identify physiological data based on bio-markers associated with the user's respiratory system.

As the filtration insert 440 and/or the inner textile layer 450 may be removable from the mask body 420, in some situations, the user may find it beneficial to be able to replace the filtration insert 440 after prolonged use and/or replace the inner textile layer 450 after prolonged use. As the inner textile layer 450 may be nearest to the user's nasal-oral cavity, it may be beneficial when the inner textile layer 450 is replaceable to maintain hygiene care.

In some embodiments, the filtration insert 440 or the inner textile layer 450 may be have a physical shape that may correspond to a contour profile of the user. For instance, the contour profile may correspond to a contoured profile spanning the nasal-oral cavity/region of the user.

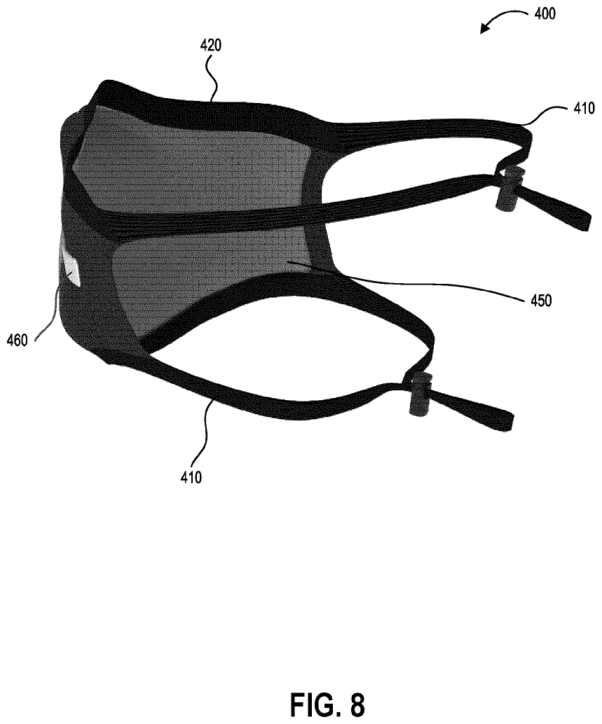

Reference is made to FIG. 8, which illustrates a rear perspective view of the mask 400 of FIG. 4, in accordance with an embodiment of the present disclosure. In FIG. 8, the filtration insert 440 (not explicitly illustrated in FIG. 8) may be received between the mask body 420 and the inner textile layer 450. In FIG. 8, the mask body 420, the filtration insert 440, and the inner textile layer 450 is illustrated in an assembled form as a combined whole.

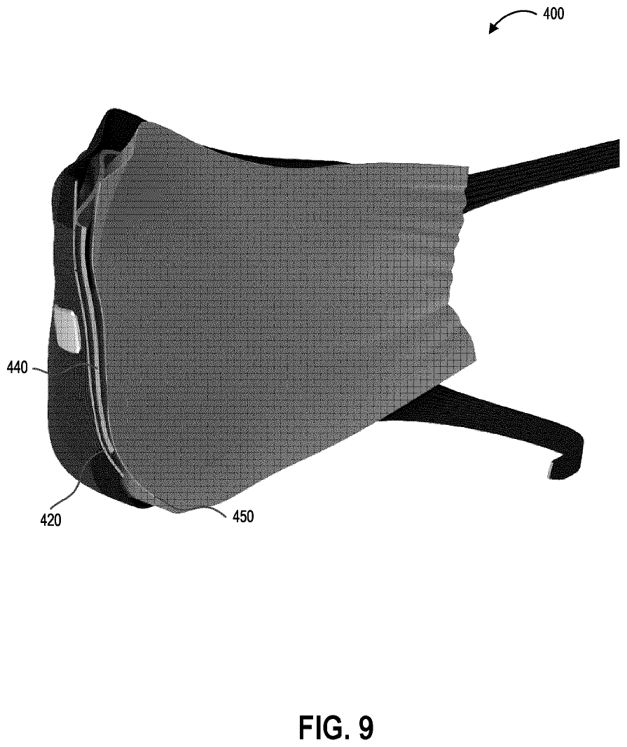

Reference is made to FIG. 9, which illustrates a cut-away, rear-perspective view of the mask 400 illustrated in FIG. 8. In FIG. 9, the mask body 420, the filtration insert 440, and the inner textile layer 450 is illustrated in assembled form. The cut-away view of FIG. 9 illustrates the positioning of the respective layers relative to other layers.

In some embodiments, one or more of the mask body 420, the filtration insert 440, and the inner textile layer 450 may include contours corresponding to a user's nose, mouth/lips, and/or the nasal-oral cavity/region more generally.

In some embodiments, the filtration insert 440 may have a smaller surface area relative to the mask body 420 or the inner textile layer 450. In some configurations, the filtration insert 440 may be dimensioned or sized to be nested between the mask body 420 or the inner textile layer 450.

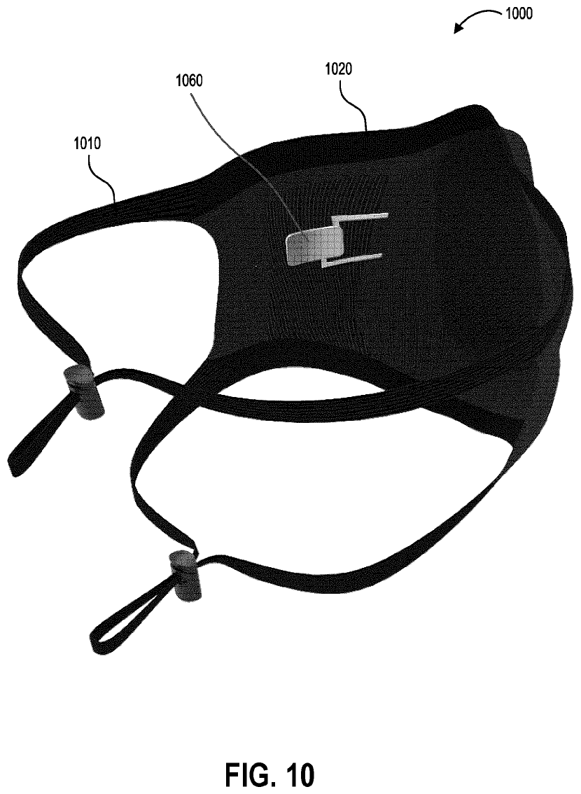

Reference is made to FIG. 10, which illustrates a rear perspective view of a mask 1000, in accordance with an embodiment of the present disclosure. The mask 1000 includes an attachment member 1010 and a mask body 1020 coupled to the attachment member 1010. The attachment member 1010 may be adapted to retain the mask body 1020 adjacent the nasal-oral cavity of the user when the mask 400 is worn by the user. In some embodiments, the mask 1000 may include similar features as the mask 400 illustrated in FIG. 4. For instance, the mask body 1020 may include a conductive fiber network electrically coupling a respiration region and a peripheral region of the mask body 1020. The mask body 1020 may include a textile with electrically conductive fibers.

In some embodiments, the mask 1000 may include a computing device 1060 coupled via the conductive fiber network to the textile mask body 1020. The computing device 1060 may include a processor and memory coupled to the processor. The memory may store processor executable instructions that, when executed, configure the processor to detect sensor data from sensory structures of the mask 1000. In some embodiments, the memory may store processor executable instructions that, when executed, configure the processor to transmit actuating signals to electro-mechanical structures for altering physical properties or electrical properties of the mask 100.

In some embodiments, the mask 1000 may include a power source device (not explicitly illustrated in FIG. 10). The power source device may be attached to the attachment member 1010 and, when the mask 1000 is worn by the user, the power source device may be positioned or tucked behind the ear of the user. In some embodiments, the power source device may be coupled to or integrated with the computing device 1060. The power source device may provide power to the computing device 1060 and/or provide power to the sensory structures or actuator structures positioned across the textile mask body 1020. The power source device may deliver power to the sensory structures or actuator structures via the conductive fiber network. In some embodiments, the mask 1000 may include a power interface or modality for interfacing/connecting to one or more other garments having an existing power delivery network integrated thereon.

In some embodiments, the textile mask body 1020 may include piezo-electric fibers configured as energy-harvesting structures. When configured as energy-harvesting structures, in response to movement caused by user respiration (e.g., inhale/exhale), deformation of the piezo-electric fibers may generate power. The generated power may be transmitted via the conductive fiber network for storage at the power source device. In some embodiments, the piezo-electric fibers may be configured to generate a sufficient quantity of power to supply power to the computing device, sensory structures, and/or actuator structures of the mask. In some embodiments, the power harvesting or generating features of the mask 1000 may include a triboelectric nano-generator.

In some embodiments, the computing device may include one or more movement sensors, such as an accelerometer, gyroscope, magnetometer may, or the like. The computing device may conduct operations to receive or detect movement data of the mask user and may associate the movement data to other sensor data detected by sensory structures associated with the respiratory region and/or the peripheral region of the textile mask body 1020. In some situations, the computing device may conduct operations for correlating a user's physiological changes to movement data of the user.

In some situations, when a user wears the textile mask body 1020, the user's voice may be muffled. In some embodiments, the textile mask body 1020 may include at least a microphone device for transmitting signals for output at a loudspeaker that may be integrated on the textile mask body 1020 or may be remote from the textile mask body 1020 for amplifying the user's voice.

Examples of system feedback features will be described with reference to the mask 100 of FIG. 1. It will be understood that embodiment features described herein may be provided by one or more of the embodiment masks described in the present disclosure.

### System Feedback: Temperature Regulation

In some embodiments, the mask 100 may be configured to provide temperature regulation features. For example, the respiration region 122 may include one or more sensory structures adapted to sense and identifying relative temperature within the nasal-oral region of the user. Further, the respiration region 122 or the peripheral region 124 may include electro-mechanical structures, such as shape-shifting structures or yarns, adapted to alter the environmental conditions within the textile mask body 120 when worn by the user.

The computing device may conduct operations to detect, via one or more environment sensors, temperature changes or humidity changes within the nasal-oral region of the user when the mask is worn by the user. In response to identifying temperature changes or humidity changes beyond a threshold value, the computing device may conduct operations to alter a state of the shape-shifting material to promote air flow or to increase moisture wicking away from the environment within the textile mask body 120.

### System Feedback: Temperature Regulation Internal to External Comparison

In some embodiments, the textile mask body 120 may include one or more sensory structures positioned on an outer layer or surface adapted to detect environmental conditions external to the textile mask body 120. Further, the textile mask body 120 may include one or more sensory structures on an inner layer or surface adapted to detect environment conditions internal to the textile mask body 120 (e.g., proximal to the nasal-oral region of the user when the mask is worn by the user). In response to identifying a difference in environment condition parameter that meets a threshold value, the computing device may conduct operations to alter a state of the shape-shifting material or yarns to promote air flow or to increase moisture wicking away from the environment within the textile mask body 120.

For example, the computing device may identify, based on sensory data, a temperature difference between the region interior to the textile mask body 120 and the environment external to the textile mask body 120 to be greater than 10 degrees Celsius. In another example, the computing device may identify, based on sensory data, a humidity difference between the region interior to the textile mask body 120 and the environment external to the textile mask body 120 to be greater than 25%. When differences in environment are identified, the computing device may conduct operations to provide feedback operations to alter the shape of the mask to promote environment regulation, thereby allowing the user to utilize the textile mask body for longer durations of time with increased comfort.

### User Physiological Status Monitoring

In some embodiments, the textile mask body 120 may include one or more sensory structures adapted to measure characteristics of air associated with a user's respiration. For example, the one or more sensory structures may be located within the respiration region 124 of the textile mask body 120. The one or more sensory structures may include humidity sensors, gas sensors, or other sensory structures for identifying air quality characteristics or parameters.

In some embodiments, the sensory device may be a device to detect gases such as volatile organic compounds (VOC) for monitoring the air of the user as the user breathes. In such embodiments, the sensory device may be referred to as a “digital nose.” The sensory device may identify the composition of the air, including identification of carbon monoxide, carbon dioxide, oxygen, nitrogen, or other gaseous elements. In some embodiments, the computing device may determine, based on the sensory device identification of air composition, bio-markers associated with the user. In some examples, the computing device may generate a combination data set of the near-real-time physiological data associated with the user and generate a summarized physiological report to the user based on bio-markers associated with the user's respiration. In the present example, the textile mask body 120 may be adapted to detect and generate physiological data associated with the user based on a user's respiratory activity when the user utilizes the mask 100 as a barrier between the user's respiratory system and the external environment.

### Adaptive Fit Textile Mask Body

In some embodiments, the textile mask body 120 may include one or more sensory structures adapted to identify changes to the fit of the textile mask body 120 on the user and, in response, the computing device may generate signals to alter states of shape shifting materials to increase the level of sealing between edges of the textile mask body and the user's face.

For example, the textile mask body 120 may include piezo-electric yarn fibers configured to generate electrical signals in response to structural changes to the piezo-electric yarn fibers. The computing device may conduct operations to receive the electrical signals that indicate structural changes to the textile mask body 120 as the user respires or as the structural shape of the mask changes over time due to temperature changes or air humidity changes. That is, as the textile mask body 120 becomes moist or becomes heated due to user respiration, the textile mask body 120 may change shape, thereby introducing open areas between the user and fringe or edge regions of the textile mask body 120. In response to identifying structural changes to the textile mask body 120 that reduces the effectiveness of the textile mask body 120 as a physical barrier to external substances, the computing device may generate signals to be transmitted via the conductive fiber network to change the shape of shape shifting textile structures to increase a seal between the textile mask body 120 and the user (e.g., reduce open areas between the user and the fringe or edge regions of the textile mask body 120. For example, the computing device may transmit electrical current to the shape shifting textile structures to cause the shape shifting textile structures to physically constrict or shrink in size.

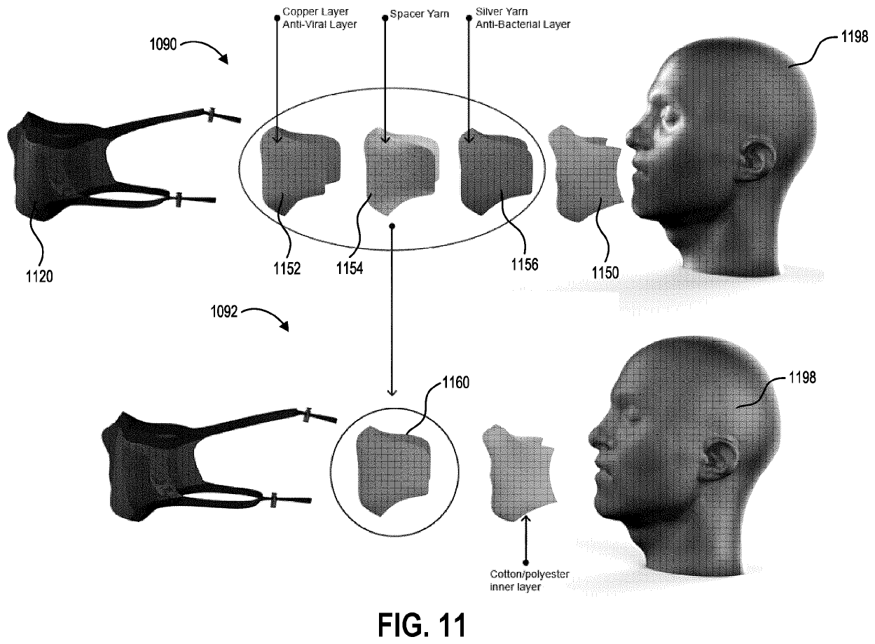

Reference is made to FIG. 11, which illustrates an exploded view 1190 and a partially exploded view 1192 of a mask 1100 adapted to be worn adjacent a nasal-oral cavity of a user 1198, in accordance with an embodiment of the present disclosure.

The mask 1100 may include a copper layer 1152, a spacer yarn layer 1154, ora silver yarn layer 1156. The copper layer 1152 may be a textile layer having copper threads knitted or otherwise integrated therein to provide an anti-viral layer. The copper threads integrated in the textile layer may provide an increased ability to act as a virus barrier. In some situations, copper-based materials may be beneficial in providing greater resistance to viruses encountering the mask 1100.

The silver yarn layer 1156 may be a textile layer having silver threads knitted or otherwise integrated therein to provide an anti-bacterial layer. The silver threads integrated in the textile layer may provide an increased ability to act as a bacteria layer. In some situations, silver-based materials may be beneficial in providing greater resistance to bacteria encountering the mask 1100.

In some embodiments, a spacer yarn layer 1154 may be positioned between the copper layer 1152 and the silver yarn layer 1156. In some embodiments, the copper layer 1152, the spacer yarn layer 1154, and the silver yarn layer 1156 may be knitted or otherwise joined together to provide a shape-shifting filter 1160 for regulating thermal or moisture at the mask 1100.

In some embodiments, copper threads (at the copper layer 1152) or silver threads (at the silver yarn layer 1156) may be coupled to a power source, such that electric current may be passed through the threads. Passing electric current through the threads may result in generated heat to the mask 1100, thereby providing heat to the user's oral-nasal cavity region. In some embodiments, passing electric current through the copper or silver threads of the respective layers may increase anti-viral or anti-bacterial properties of the respective mask layers. In some embodiments, passing electric current through fibers of one or more layers (in the combination of layers) may generate opposing or alternating poles or electric fields, thereby promoting attraction to small particles at the mask 1100 surface (e.g., trapping small particles).

In some embodiments, the combined copper layer 1152/spacer yarn layer 1154/silver yarn layer 1156 may be positioned between a textile mask body 1120 and a mask inner layer 1150. In some embodiments, the textile mask body 1120 may include an outer layer having hydrophobic polyester layer for repelling liquid or droplets that may be splashed upon the textile mask body 1120. In some embodiments, the mask inner layer 1150 may be constructed of a blend of cotton or polyester material. Other materials for the mask inner layer 1150 may be contemplated.

In some embodiments, the combination of the copper layer 1152/spacer yarn layer 1154/silver yarn layer 1156 may be coupled to a power source, and at least one or more copper fibers or silver fibers may receive electric current. In the present example, the copper or silver fibers receiving electric current may be configured to: (1) provide heating to the facial garment user's nasal-oral region (when the facial garment is worn by the user); and (2) increase anti-viral/anti-bacterial resistant properties of the facial garment when the electric current may be passing through at least one of the copper or silver fibers.

In another embodiment, at least one of the copper layer 1152, spacer yarn layer 1154, or silver yarn layer 1156 may include fibers configured to sense temperature proximal to the facial garment user or to sense temperature on an exterior facing surface of the facial garment. For example, fibers that may sense temperatures may exhibit changes in resistive properties when sensed temperatures change. Thus, in the present example, when at least one fiber in the combination of layers receives electric current, the combination of layers may be configured to: (a) provide heating to the facial garment user's nasal-oral region; (b) increase anti-viral/anti-bacterial properties of the facial garment when the electric current may be passing through at least one fiber of the combination of layers; and (c) provide temperature sensing features based on changes in electrical properties based on resistive property changes of fibers.

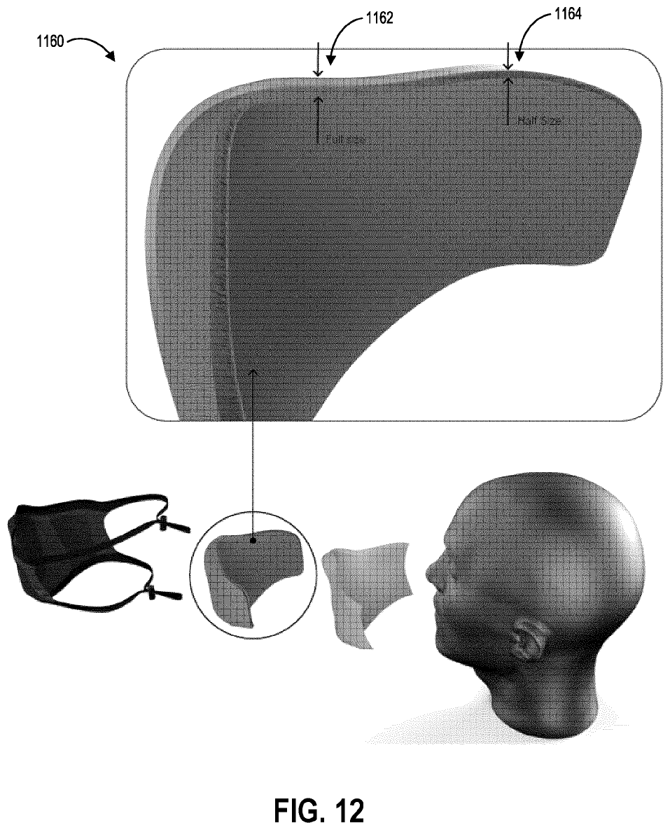

Reference is made to FIG. 12, which illustrates an enlarged, cutaway view of the shape-shifting filter 1160 illustrated in FIG. 11. In some embodiments, the shape-shifting filter 1160 may include a combination of the copper layer 1152, the spacer yarn layer 1154, and the silver yarn layer 1156 of FIG. 11.

The shape-shifting filter 1160 may be configured to transition between a first thickness 1162 and a second thickness 1164 at a given position of the shape-shifting filter 1160 in response to changes in at least one of temperature, humidity, or other impetus. In some embodiments, the shape-shifting filter 1160 may have varying thickness about the perimeter of the filter because the various positions may experience changes in at least one of temperature, humidity, or other environmental factor at different rates.

In some embodiments, the shape-shifting filter 1160 may include fibers coupled to a power source, and electric current passed through one or more fibers may, at least in part, cause changes in the overall thickness of the shape-shifting filter 1160.

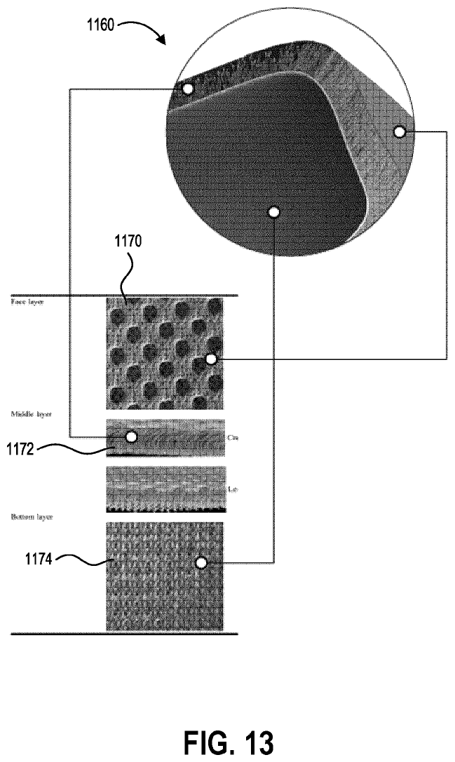

Reference is made to FIG. 13, which illustrates an enlarged, partial cutaway view of the shape-shifting filter 1160 of FIG. 11. The enlarged, partial cutaway view further provides an enlarged view of details of the various layers of the shape-shifting filter 1160. In some embodiments, the shape-shifting filter 1160 may include a face layer 1170 having one or more layers of porous textile, where the layers of porous textile may have one or more pore sizes. In some embodiments, the middle layer 1172 of the shape-shifting filter 1172 may include yarn fibers, or the like. In some embodiments, the shape-shifting filter 1160 may include a bottom layer 1174, having surface details illustrated in FIG. 13. In some embodiments, one or more of the layers of the shape-shifting filter 1160 may include silver or copper fibers knitted or otherwise integrated therein.

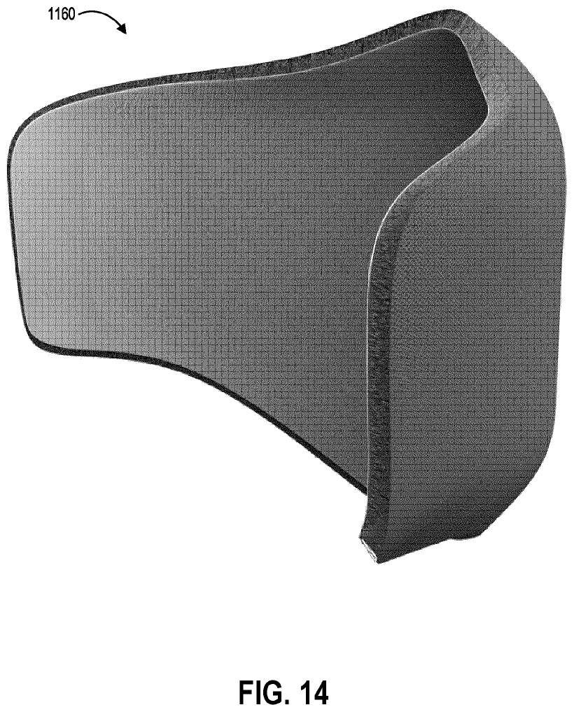

Reference is made to FIG. 14, which illustrates a perspective view of the shape-shifting filter 1160 of FIG. 11.

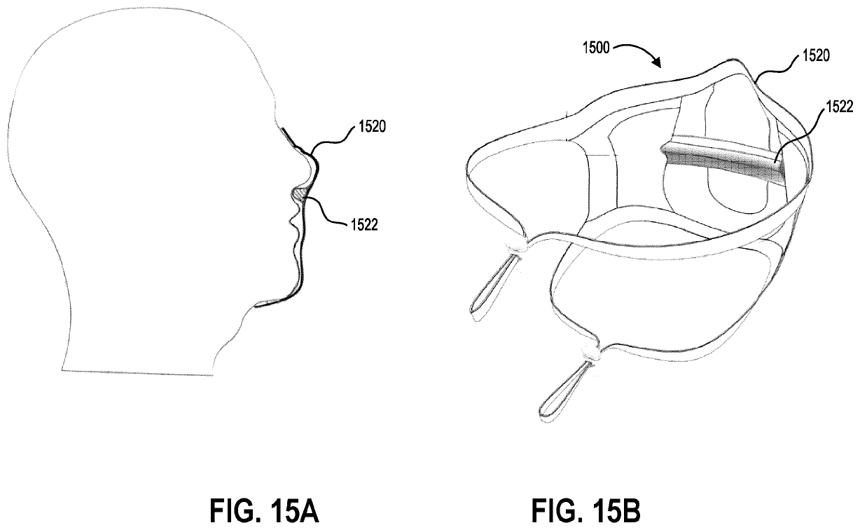

Reference is made to FIG. 15A, which illustrates a side, cross-sectional view of a textile mask body 1520 positioned about an oral-nasal cavity region of a user, in accordance with an embodiment of the present disclosure. The textile mask body 1520 may include features similar to one or more embodiments of textile mask body examples described in the present disclosure.

The textile mask body 1520 may include a spacer structure 1522. The spacer structure 1522 may be affixed or positioned on the textile mask body 1520 at a position such that the spacer structure 1522 provides “micro-climates” to the user when the textile mask body 1520 is worn by the user. For example, the spacer structure 1522 may provide at least a partially separated region to separate air in the nasal region of the user from air in the oral region of the user. In scenarios where the user may inhale via the user's nose and exhale via the user's mouth, or vice versa, the spacer structure 1522 may reduce situations where the user inhales air that was recently exhaled.

Reference is made to FIG. 15B, which illustrates a rear perspective view of a facial garment 1500, in accordance with an embodiment of the present disclosure. The facial garment 1500 may include features similar to one or more embodiments of facial garment examples described in the present disclosure. The facial garment 1500 may include the spacer structure 1522 that may be positioned between a user's nose and mouth when the facial garment 1500 is worn by the user.

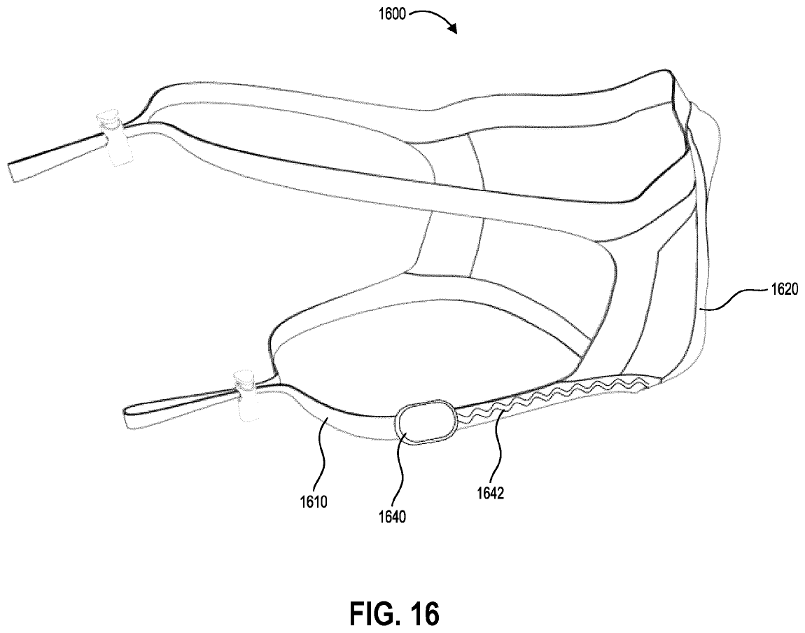

Reference is made to FIG. 16, which illustrates a perspective view of a facial garment 1600, in accordance with an embodiment of the present disclosure. The facial garment 1600 may include a computing device 1640 affixed to a portion of the attachment member 1610. The computing device 1640 may be coupled via a fiber network 1642 to one or more sensor or actuator devices (not explicitly illustrated in FIG. 16). The one or more sensor or actuator devices may be knitted or otherwise integrated on the mask body 1620 for detecting sensory input from the user when the facial garment 1600 may be worn by the user or for outputting actuating signals at the facial garment 1600 for adapting the facial garment 1600 to detected environment factors. In some embodiments, the fiber network 1642 for coupling the computing device 1640 to one or more sensor or actuator devices may be a two wire fiber network. Other configurations of the fiber network 1642 may be contemplated.

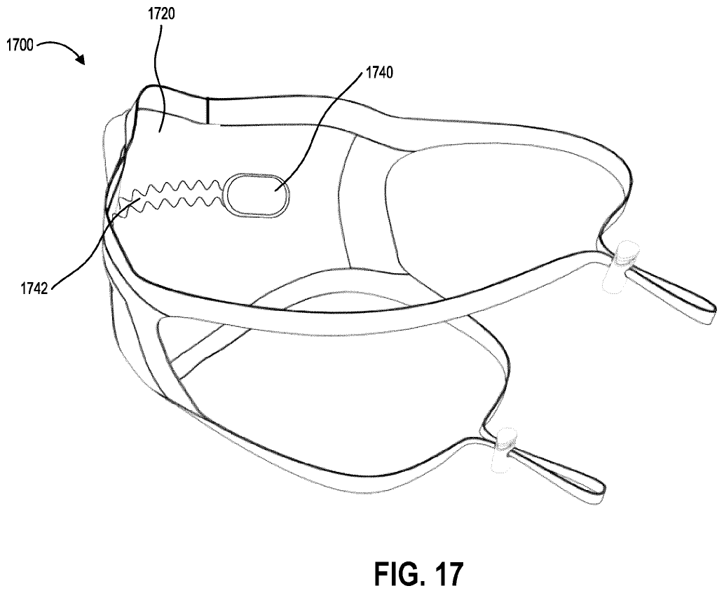

Reference is made to FIG. 17, which illustrates a perspective view of a facial garment 1700, in accordance with an embodiment of the present disclosure. The facial garment 1700 may include a computing device 1740 affixed to an interior surface or an exterior surface of a textile mask body 1720. The computing device 1740 may be coupled via a fiber network 1742 to one or more actuators or sensors knitted or otherwise integrated with the textile mask body 1720.

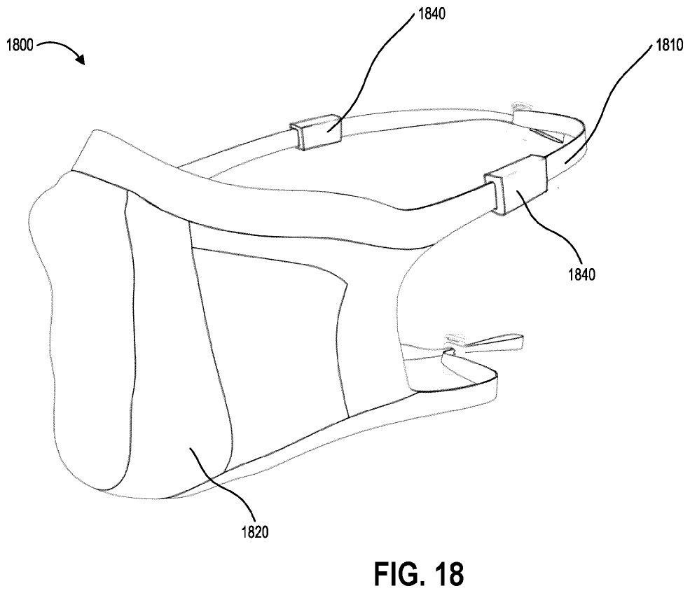

Reference is made to FIG. 18, which illustrates a perspective view of a facial garment 1800, in accordance with an embodiment of the present disclosure. The facial garment 1800 may include one or more electronic devices 1840 coupled to at least a portion of an attachment member 1810. The one or more electronic devices 1840 may be coupled, via a fiber network integrated within or atop the attachment member 1810, to one or more sensors or actuators knitted or otherwise integrated in a mask body 1820.

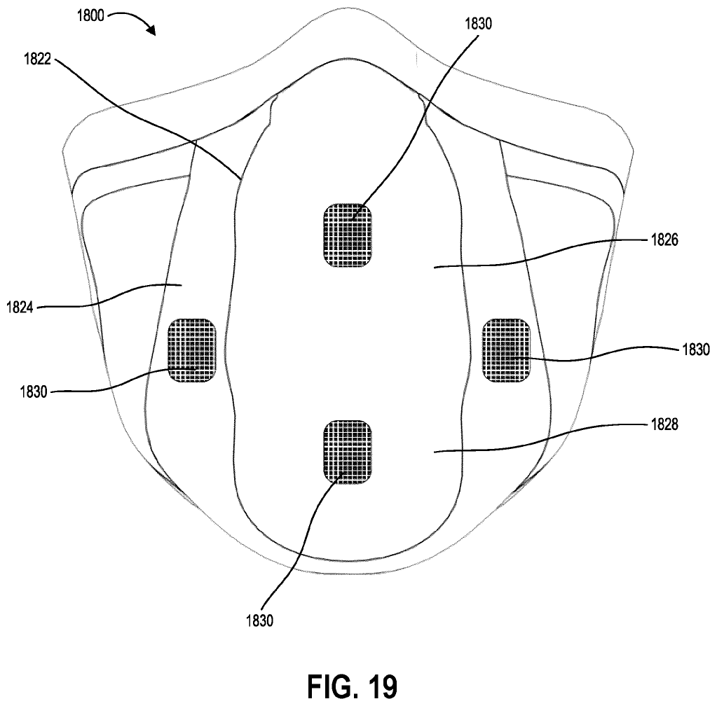

Reference is made to FIG. 19, which illustrates a rear plan view of the facial garment 1500 illustrated in FIG. 15. The facial garment 1500 may include a respiration region 1822 adapted to cover a portion of an oral-nasal region of a user when the mask is worn by the user. The respiration region 1822 may include a nasal sub-region 1826 and an oral sub-region 1828. The facial garment 1500 may include a peripheral region 1824 that may be adjacent to at least a portion of the respiration region 1822.

In some embodiments, one or more sensors or actuators 1830 may be knitted or integrated in the facial garment 1500 at one of the above-described regions to detect or gather biometric or physiological data of the user. The illustrated positioning of the one or more sensor or actuators 1830 in FIG. 19 is exemplary, and other positioning configurations may be contemplated. In some embodiments, the one or more sensors or actuators 1830 may be off-the-shelf devices that may be coupled to the facial garment at particular placement positions.

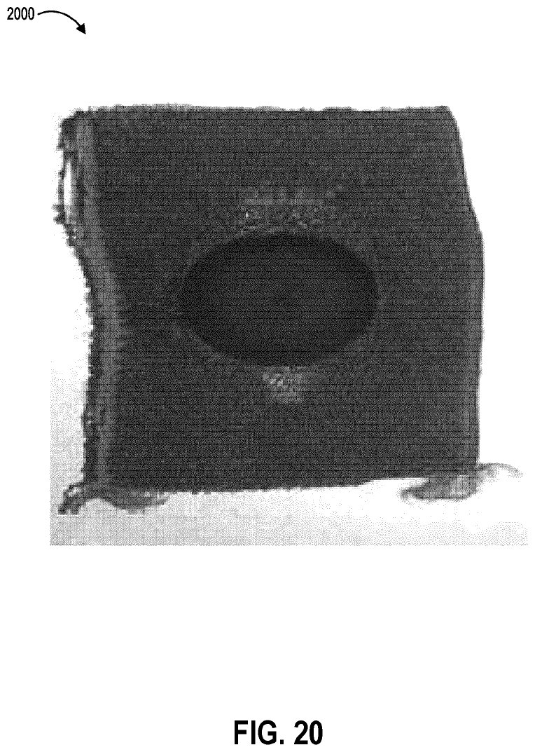

FIG. 20 illustrates a top view of a sensor or actuator 2000 knitted or integrated in a textile, in accordance with an embodiment of the present disclosure.

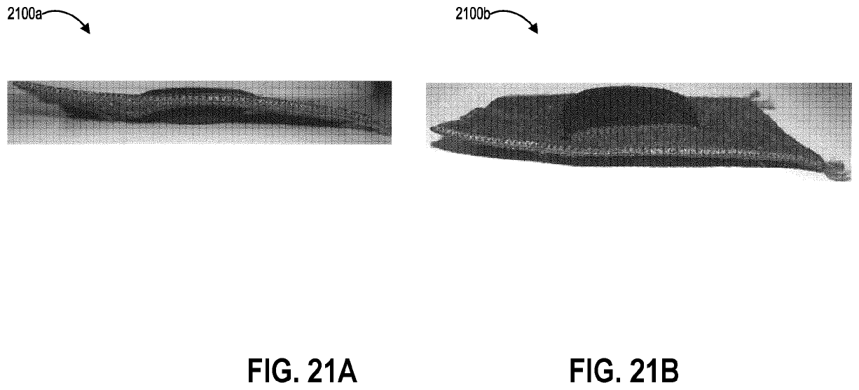

FIG. 21A illustrates a side elevation view of a sensor or actuator 2100a knitted or integrated into a textile, in accordance with an embodiment of the present disclosure.

FIG. 21B illustrates a perspective view of a sensor or actuator 2100b knitted or integrated into a textile, in accordance with an embodiment of the present disclosure.

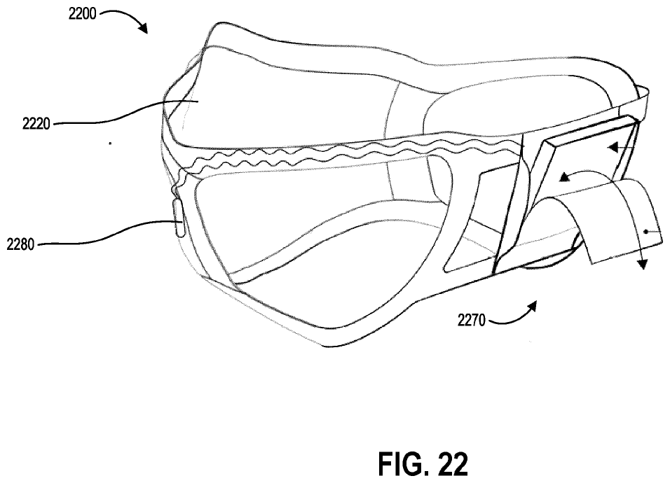

Reference is made to FIG. 22, which illustrates a rear perspective view of a facial garment 2200, in accordance with an embodiment of the present disclosure. The facial garment 2200 may include a rear compartment 2270 for receiving at least one of an electronic module, a power supply, or a communication transceiver. In some embodiments, the electronic module may be coupled, via a fiber network knitted or integrated in an attachment member, to a sensor module 2280 that may be coupled to a textile mask body 2220.

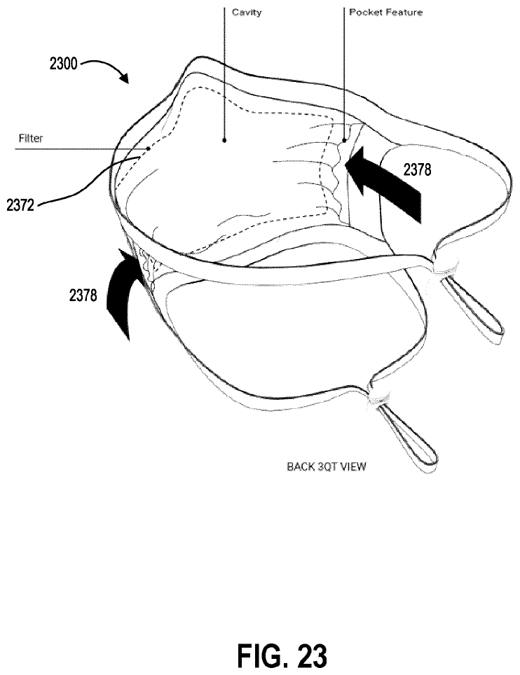

FIG. 23 illustrates a rear perspective view of a facial garment 2300, in accordance with an embodiment of the present disclosure. The facial garment 2300 may include at least one pocket insertion opening 2378 at a fringe portion of the facial garment 2300 for insertion or removal of a filtration insert 2372.

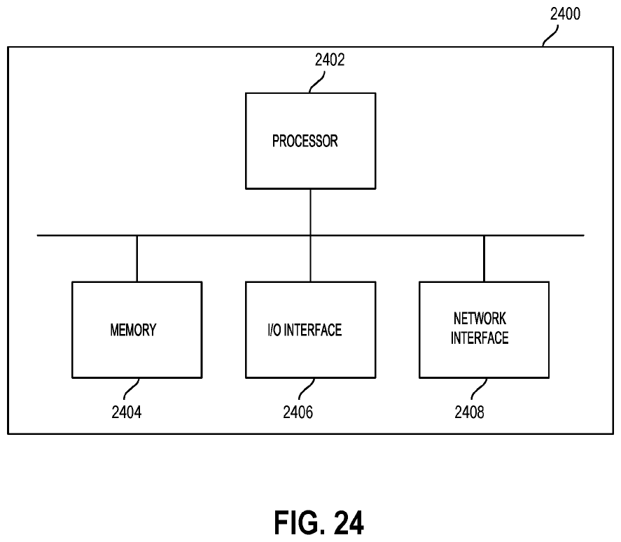

Reference is made to FIG. 24, which illustrates a block diagram of a computing device 2400, in accordance with an embodiment of the present disclosure. As an example, the computing device 2400 may be implemented as an “e-module” illustrated in some drawings of the present disclosure or as a computing device coupled to one or more sensors or actuators for detecting physiological or environment data or providing actuating signals to textile fibers or actuators integrated thereon.

The computing device 2400 may include at least one processor 2402, memory 2404, at least one I/O interface 2406, and at least one communication circuit 2408.

The processor 2402 may be a microprocessor or microcontroller, a digital signal processing (DSP) processor, an integrated circuit, a field programmable gate array (FPGA), a reconfigurable processor, a programmable read-only memory (PROM), or combinations thereof.

The memory 2404 may include a computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM).

The I/O interface 2406 may enable the computing device 2400 to interconnect with one or more input devices, such as a keyboard, mouse, camera, touch screen and a microphone, or with one or more output devices such as a display screen and a speaker.

The communication circuit 2408 may be configured to receive and transmit data sets to or from one or more sensors or actuators coupled to a facial garment or textile, in accordance with embodiments of the present disclosure.

Facial garments may include masks, among other examples, for covering at least a nasal-oral region of a user's face. When a facial garment is fitted to a user's head, the facial garment may impede air flow to the user's face and the user may breathe in substantially the same air that the user recently breathed out (e.g., a user breathing in air that has been recently exhaled and trapped within the facial garment). It may be beneficial to provide facial garments that may reduce inhalation of recently exhaled air by a user.

As facial garments may be worn to provide a physical barrier between a user's nasal-oral region and the environment, it may be beneficial to provide facial garments with filtration devices. It may also be beneficial to provide facial garments with sensor devices for assessing user health or user performance based at least on emissions from the user's nasal-oral region. For example, it may be beneficial to provide connected garments proximal to a user's nasal oral region for generating, via volatile organic compound sensing among other examples, user data based on emissions from a user's respiratory system.

In some scenarios, it may be beneficial to combine facial garment sensing features with garment sensing features associated with other portions of a user's body. For example, it may be beneficial to generate a status indication of a user's health based on a combination of biochemical, biomechanics, electrophysiology, and haemodynamic data.

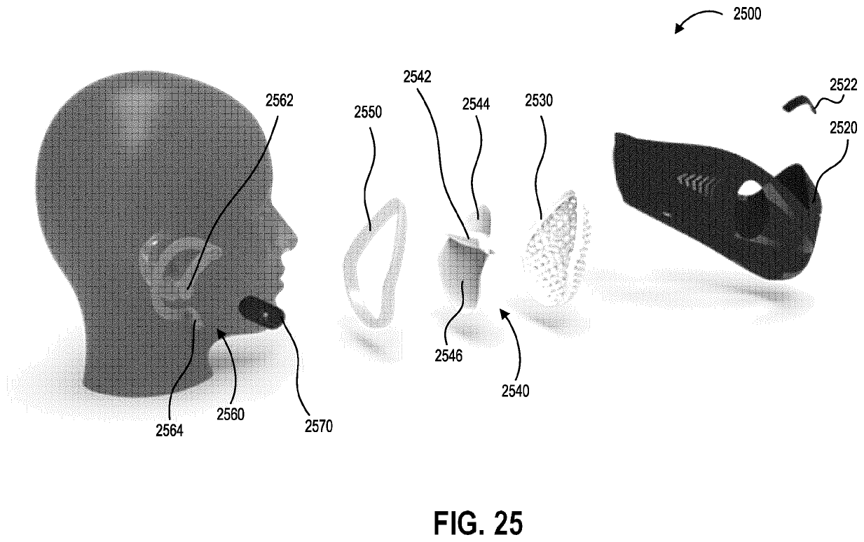

To illustrate embodiments of facial garments, reference is made to FIG. 25. FIG. 25 illustrates an exploded, rear perspective view of a mask 2500, in accordance with an embodiment of the present disclosure. The mask 2500 may be adapted to be worn over the mouth and the nose of a user. When worn over the user's nasal-oral region, the mask 2500 may include features for sensing emissions from the user's respiratory system. In some embodiments, the mask 2500 may include features for reducing inhalation of recently exhaled air by the user.

The mask 2500 includes a textile mask body 2520. The textile mask body 2520 may be configured to be fitted against a nasal-oral region of the user. The textile mask body 2520 may include a network of natural or synthetic fibers. The textile mask body 2520 may include an attachment portion configured to wrap around a user's head. The attachment portion may include one or more apertures 2524 for receiving an ear of the user. In the illustrated embodiment of FIG. 25, the attachment portion configured to wrap around a user's head may reduce tension or stress that may otherwise be applied to the user's ears for retaining the mask 2500 to the user's oral-nasal region.

The mask 2500 may include a nose clip 2522. The nose clip 2522 may be a malleable member configured to shape a portion of the textile mask body 2520 along the contour of the user's nose.

The mask 2500 may be modular and may include a combination of components integrated to or held together by the textile mask body 2520. The mask 2500 includes a filtration device 2530. The filtration device 2530 may include an N95 filtration insert, a coper-treated nylon insert, a BIOSA enzyme-contained film inert, a non-woven sheet insert, or a combination of any thereof for providing a filtering barrier between the user's nasal-oral region and the environment. Other types of filtration insert materials may be contemplated.

The mask 2500 may include a nasal-oral divider member 2540 configured to provide at least partially separated regions to separate a quantity of air in the nasal region of the user and air in the oral region of the user. The partially separated regions may be configured by a spacer structure 2542. The spacer structure 2542 may separate a nasal region 2544 and an oral region 2546 of the nasal-oral divider member 2540. When the mask 2500 is worn by a user, the nasal-oral divider member 2540 may reduce inhalation of recently exhaled air by a user.

The mask 2500 includes a seal member 2550. The seal member 2550 may be a malleable or flexible member configured to reduce gaps between the textile mask body 2520 and the user's face about the nasal-oral region. In some embodiments, the seal member 2550 may be constructed of silicone or other malleable materials.

One or more of the filtration device 2530, the nasal-oral divider member 2540, or the seal member 2550 may be combined for retaining against the oral-nasal region of the user by the textile mask body 2520.

In some embodiments, the textile mask body 2520 may include yarns with heating elements, yarns having electrostatic properties, or yarns having temperature sensing features. The textile mask body 2520 may include resistive fibers configured to increase temperature about the nasal-oral region of the user. In some embodiments, the textile mask body 2520 may include silver or copper yarns knitted therein to provide an anti-microbial or anti-viral barrier between the user and the user's environment. When power is provided to the resistive fibers, the resistive fibers may be configured to increase the temperature about the nasal-oral region. Elevated temperature may promote release of ions from copper or silver yarns for counteracting or eradicating undesirable properties of viruses, among other foreign substances. The textile mask body 2520 may include yarns having electrostatic properties that may be configured to provide a barrier to virus or bacteria.

In some embodiments, the textile mask body 2520 may include temperature sensing yarns or fibers. The textile mask body 2520 may be configured to detect changes in temperature based on inhalation or exhalation by the user and, by proxy, to detect breathing characteristics such as respiration rate.

In some embodiments, fibers/yarns or other devices integrated on or coupled to the textile mask body 2520 may be coupled to an on-mask computing device 2570. The on-mask computing device 2570 may include a power source for supplying power to fibers/yarns or other devices integrated on or coupled to the textile mask body 2520. In some embodiments, the textile mask body 2520 may include data communication fibers for transmitting data sensing signals to the on-mask computing device 2570. In some embodiments, the on-mask computing device 2570 may store or combine the plurality of data sensing signals, and may transmit the plurality of data sensing signals to a nearby computing server (e.g., user mobile device, personal computing device, etc.) for processing. The on-mask computing device 2570 may transmit the plurality data signals to a nearby computing server via a communication transceiver, such as a Bluetooth™ Low Energy transceiver, among other communication transceiver devices. In some embodiments, the communication transceiver may be other types of near-field communication transceivers.

In FIG. 25, the on-mask computing device 2570 may be positioned on a right-side of the textile mask body 2520. In some other embodiments, the on-mask computing device 2570 may be positioned on a left-side of the textile mask body 2520 or at other positions (e.g., proximal to the rear of the user's head, etc.).

In some embodiments, the textile mask body 2520 may include one or more further sensor devices. For example, the textile mask body 2520 may include integrated volatile organic compound (VOC) sensors, sweat or saliva sensors, carbon dioxide sensors, or other sensor devices for identifying characteristics of emissions from the respiratory system of the user. In some embodiments, the integrated VOC sensor may be configured to detect concentrations of gases associated with undesirable air quality. In some examples, the integrated VOC sensor may be configured to detect potential airborne infectious agents.

In some embodiments, the carbon dioxide sensors integrated in the textile mask body 2520 may be configured for generating VO2 max metrics. VO2 max may be associated with a maximum amount of oxygen associated with aerobic endurance or cardiovascular performance of a user. In some embodiments, sensors integrated in the textile mask body 2520 may be associated with determining respiratory exchange ratio (RER) of a user.

In some embodiments, the mask 2500 may be configured to include one or more auxiliary sensors 2560. In some embodiments, the mask 2500 may include an infrared sensor 2562 (e.g., IR sensor) for detecting infrared radiation, and thereby sensing the user's body temperature. In some embodiments, the mask 2500 may be configured to position the infrared sensor proximal to or partially within the ear of the user for detecting body temperature. In some embodiments, an “over the ear” bracket may be configured to position or retain the IR sensor proximal to the ear of the user. The positioning of infrared sensor allows for body temperature to be monitored continuously when the mask 2500 is worn.

In some embodiments, the textile mask body 2520 may include a photoplethysmography (PPG) sensor configured to generate optical measurements. In some embodiments, the PPG sensor 2564 may be configured to detect or determine heart rate variability (HRV) statistics, oxygen saturation (SpO2) statistics, or other heart rate monitoring statistics of the user over time. In some embodiments, the PPG sensor may be coupled to the “over the ear” bracket and be configured to position the PPG sensor proximal to the user's ear. Other placement positions of the PPG sensor about the user's face may be contemplated.

In some embodiments, the mask 2500 may be configured to be modular, and the mask 2500 may be disassembled or reassembled for hand washing, disinfecting, autoclaving, or other operations for maintaining components of the mask 2500.

In some embodiments, data detected or generated via features of the mask 2500 described herein may be combined by the on-mask computing device 2570 for transmission to an external computing server for analysis or data collection. In some embodiments, data detected or generated via features of the mask 2500 may be associated with biochemical status of the user and may be processed or combined with other physiological data of the user. For example, the mask 2500 may be configured to detect biochemical data of the user over time, and the on-mask computing device 2570 or the external computing server may combine or associate the biochemical data with detected biomechanical, electrophysiological, or haemodynamic data of the user over time for generating multi-faceted physiological data sets associated with the user. In some embodiments, biomechanical electrophysiological, haemodynamic, or other physiological data associated with the user may be generated based on textile computing systems associated with garments for other portions of the user's body. The on-mask computing device 2570 or other external computing servers may combine or generate the multi-faceted physiological data sets and may transmit signals for communicating the multi-faceted physiological status of the user. In some embodiments, generating the multi-faceted physiological status of the user based on the plurality of data generated by one or more textile computing garments may be based on machine learning operations to provide insights on user health or user performance.

In some embodiments, the external computing server may receive physiological data based on one or more garments associated with respective users of a user group. The external computing server may be configured to monitor physiological status of the user group. As an illustrating example, where members of a team respectively wear textile computing garments (e.g., embodiment facial garments described herein), the external computing server may monitor collective or respective individual physiological status of users within a group at a hospital or other workplace. In some scenarios, the on-mask computing device 2570 or the external computing server may be configured to deduce or identify symptoms of stress or fatigue associated with the user based on changes to detected respiration, body temperature, or other physiological data determined based on sensors or fibers integrated with the mask 2500. In some scenarios, detection and interpretation of physiological data of users may be beneficial for advanced identification of potential health-related outbreaks affecting health, well-being, or productivity of users. For example, the mask 2500 may be configured to be a component of a connected personal protective equipment (PPE) system utilizing volatile organic compound sensing, thereby enabling methods of holistically assessing health and performance as part of expanding interconnected systems of biometric garments.

In some embodiments, the on-mask computing device 2570 may include one or more embedded inertial measurement unit (IMU) sensors to detect or generate biomechanical data associated with the user. The on-mask computing device 2570 may correlate biochemical status data with detected biomechanical data to provide a more comprehensive correlation of user physiological status over time. For example, the on-mask computing device 2570 may provide a physiological status of the user during times before, during, or after an exercise/work-out session.

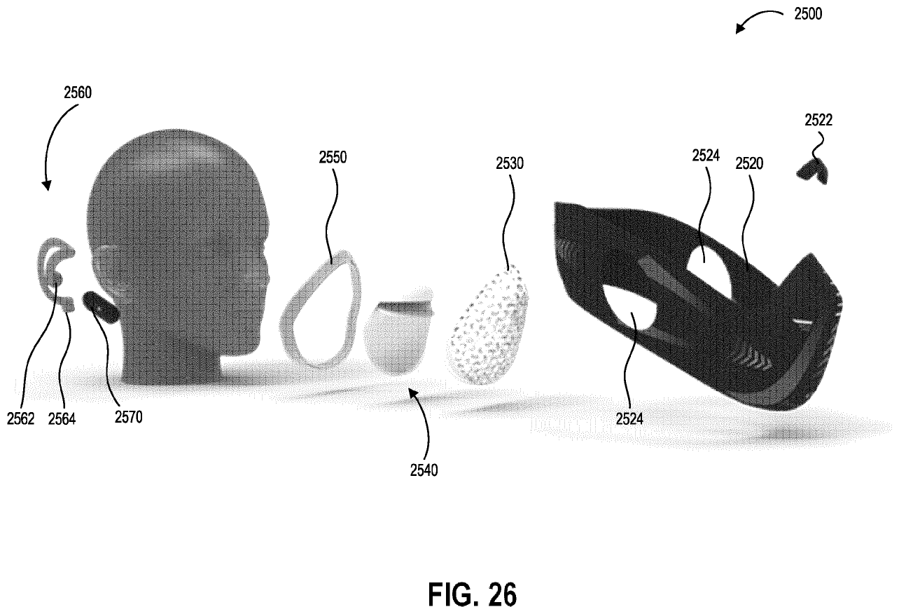

Reference is made to FIG. 26, which illustrates an exploded, front perspective view of the mask 2500 of FIG. 25. FIG. 26 illustrates apertures 2524 integrated in the textile mask body 2520 for receiving respective ears of the user. The textile mask body 2520 may include a wraparound attachment portion for fitting around a user's head, thereby reducing tension or stress that may otherwise be applied to the user's ears for retaining the mask 2500 at the user's oral-nasal region.

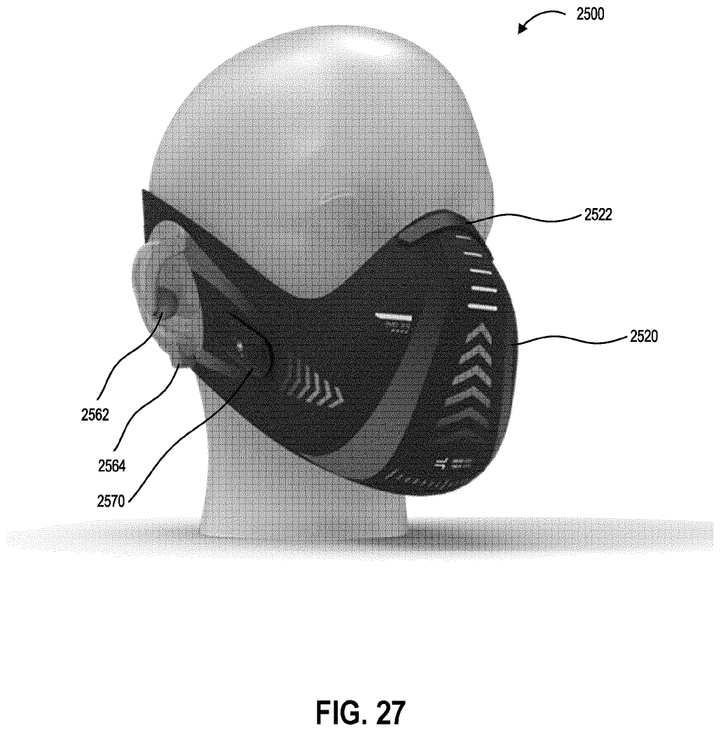

Reference is made to FIG. 27, which illustrates a front perspective view of the mask 2500 of FIG. 25 fitted to a user's head.

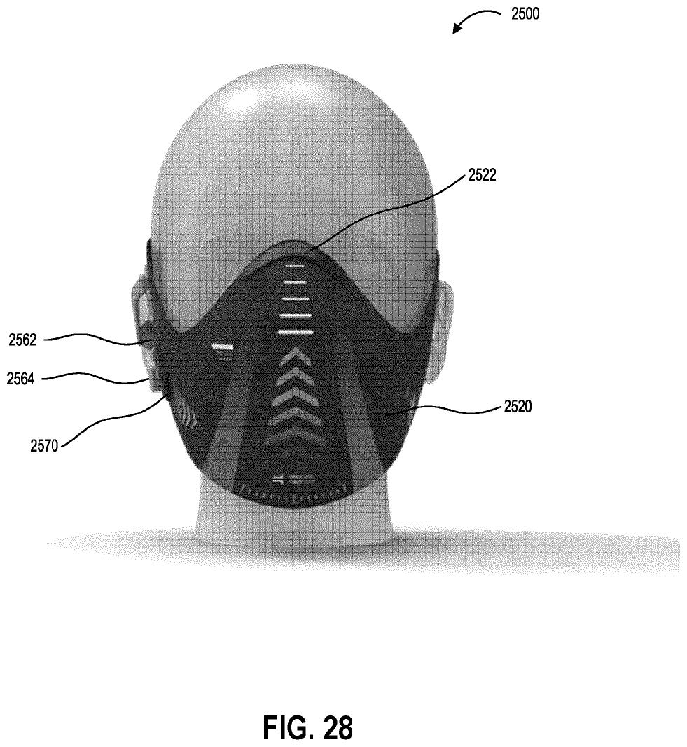

Reference is made to FIG. 28, which illustrates a front elevation view of the mask 2500 of FIG. 25 fitted to a user's head. In some embodiments, the textile mask body 2520 may be configured to align or follow with contours of the user's head. For example, the textile mask body 2520 may include contours for positioning around the user's eyes.

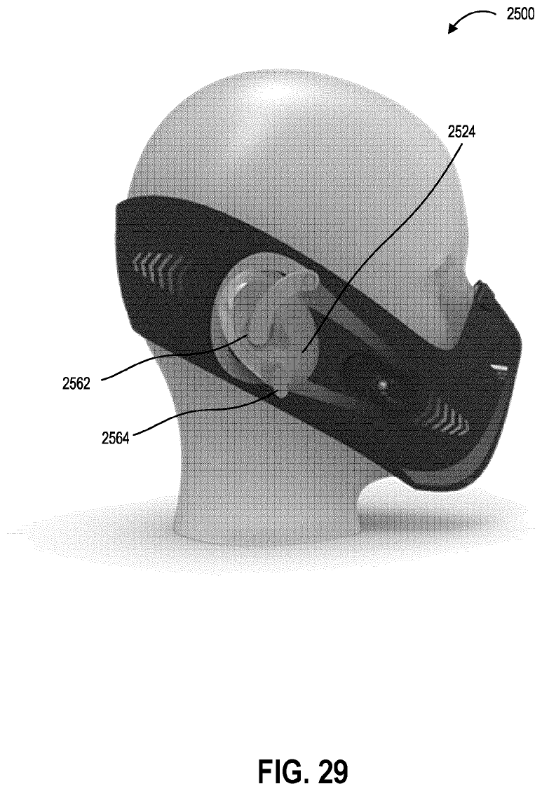

Reference is made to FIG. 29, which illustrates a right side elevation view of the mask 2500 of FIG. 25 fitted to a user's head. In some embodiments, the one or more auxiliary sensors, including one or more of the infrared sensor 2562 or the PPG sensor 2564 may be configured to be positioned proximal to the user's right ear. That is, the one or more auxiliary sensors may be configured on a right side of the textile mask body 2520.

In some other embodiments, the one or more auxiliary sensors may be configured to be positioned proximal to the user's left ear. That is, the one or more auxiliary sensors may be configured on a left side of the textile mask body 2520. Positional placements of one or more auxiliary sensors may be configured based on user preferences or regulatory standards.

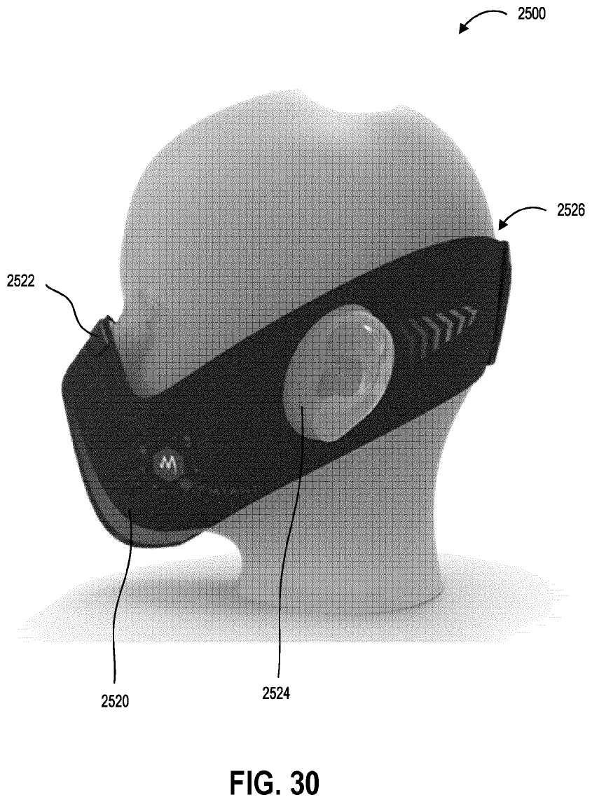

Reference is made to FIG. 30, which illustrates a left side elevation view of the mask 2500 of FIG. 25 fitted to a user's head. In some embodiments, the attachment portion may include a closure member 2526 having an attached mode and an unattached mode. For example, the closure member 2526 may be a hook-and-loop fastener member, snap-button fastening member, or other fastening members to removably attach or un-attach portions of the textile mask body 2520, thereby providing a user with mechanisms to fasten or loosen the mask 2500 from the user's head.

In some embodiments, the textile mask body 2520 may include regions having translucent or transparent regions, thereby allowing unobstructed views of the user's face.

In some embodiments, the mask 2500 may include devices for generating positive airway pressure when the mask 2500 is worn by a user, thereby providing a mechanism to make it easier for the user to breathe.

In some embodiments, the textile mask body 2520 may include an electroactive polymer integrated thereon for generating positive pressure within the nasal-oral region while the mask 2500 is worn by the user. The electroactive polymer may be configured to exhibit a change in size or shape when stimulated by an electric field, thereby generating vibrations or turbulence within the contained area between the user's nasal-oral region and the textile mask body 2520. The electroactive polymer may be integrated to the textile mask body 2520 and coupled to conducting fibers of the textile mask body 2520.

In some embodiments, the textile mask body 2520 may include a pump or motor device for generating positive pressure. In some embodiments, the pump or motor device may draw upon an air supply to generate positive pressure, thereby making it easier for the user to breathe. In some embodiments, the air supply may be based on the environment external to the user's nasal-oral region. When the air supply may be based on the external environment, the conduit for drawing air to create positive pressure within the nasal-oral region of the mask may include a filtration device (e.g., N95 filtration insert) through which the air supply passes.

In the above described-embodiments of devices for generating positive airway pressure when the mask 2500 is worn by the user, the seal member 2550 may provide a contained chamber within the nasal-oral region of the user for: (i) keeping foreign substances from the nasal-oral region of the user; or (ii) providing an insulated or sealed environment for generating positive airway pressure for the user.

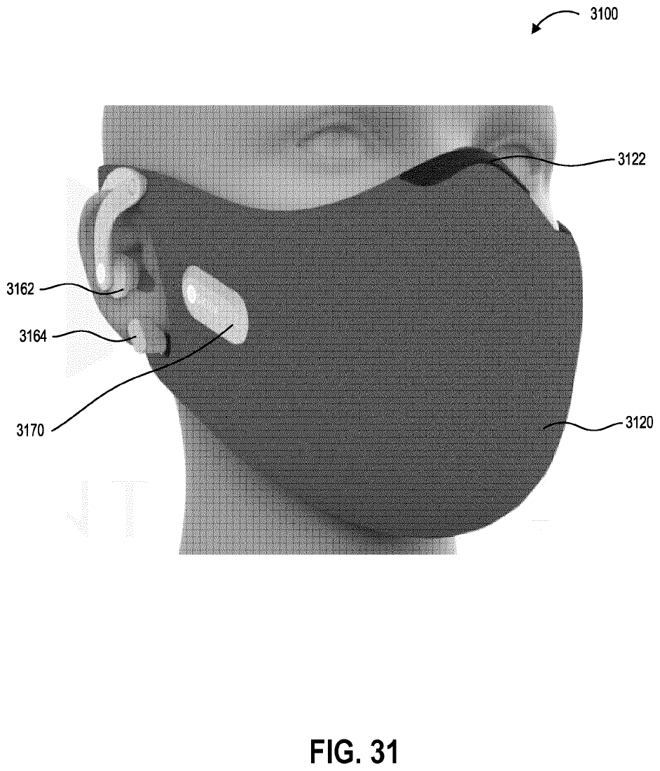

Reference is made to FIG. 31, which illustrates a front perspective view of a mask 3100, in accordance with an embodiment of the present disclosure. The mask 3100 includes a textile mask body 3120, a nose clip 3122, an infrared sensor 3162, a PPG sensor 3164, and an on-mask computing device 3170. The textile mask body 3120, the nose clip 3122, the infrared sensor 3162, the PPG sensor 3164, and the on-mask computing device 3170 may be similar to the textile mask body 2520, the nose clip 2522, the infrared sensor 2562, the PPG sensor 2564, and the on-mask computing device 2570, respectively, described with reference to FIGS. 25 to 30.

Although the embodiments have been described in detail, it should be understood that various changes, substitutions and alterations can be made herein without departing from the scope. Moreover, the scope of the present disclosure is not intended to be limited to the particular embodiments of the process, machine, manufacture, composition of matter, means, methods and steps described in the specification.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

The description provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

As can be understood, the examples described above and illustrated are intended to be exemplary only.

1. A facial garment comprising:
an attachment member for attaching the facial garment to a user;
a textile mask body coupled to the attachment member, the textile mask body including:
a respiration region adapted to cover a portion of an oral-nasal region when the garment is attached to the user, the respiratory region including a sensory structure;
a peripheral region adjacent the respiration region adapted to cover a portion of the user's cheek when the mask is worn by the user, the peripheral region including an electro-mechanical structure; and
a conductive fiber network electrically coupling the respiration region and the peripheral region; and
a computing device coupled via the conductive fiber network to the textile mask body, the computing device including a processor and a memory coupled to the processor, the memory storing processor executable instructions that, when executed, configure the processor to detect sensor data from the sensory structure and transmit actuating signals to the electro-mechanical structure.

2. The facial garment of claim 1, wherein the conductive fiber network includes at least one interface for coupling to an add-on sensory device.

3. The facial garment of claim 1, wherein the sensory structure includes an environment sensor configured to detect at least one of pressure, temperature, humidity, carbon dioxide, carbon monoxide, volatile organic compounds, or gaseous air quality gases.

4. The facial garment of claim 1, wherein the electro-mechanical structure includes shape-shifting textile to provide increased fit or comfort.

5. The facial garment of claim 1, wherein the respiration region includes a nasal sub-region adapted to cover a nasal region of the user and an oral sub-region adapted to cover an oral region of the user, and wherein the respiration region includes at least one cavity structure directing airflow among the nasal region and the oral region of the user.

6. The facial garment of claim 1, wherein the computing device includes at least one of an accelerometer, a gyroscope, or a magnetometer.

7. The facial garment of claim 1, comprising a formed pocket layer positioned at the respiration region adapted to receive a filtration insert.

8. The facial garment of claim 7, comprising at least one of an N95 filter insert, a copper-treated nylon insert, BIOSA enzyme-contained film insert, or a non-woven sheet insert removably positioned in the formed pocket layer at the respiration region.

9. The facial garment of claim 1, wherein the textile mask body includes at least one of copper or silver yarn.

10. The facial garment of claim 1, wherein the textile mask body includes hydrophobic yarn fibers on an exterior portion of the textile mask body to repel or prevent virus or bacteria infected droplets from penetrating to the interior portion of the textile mask body.

11. The facial garment of claim 1, wherein the textile mask body includes a conductive yarn including at least one of silver or copper.

12. The facial garment of claim 11, wherein the conductive yarn is configured to provide at least one of generated heat, increased anti-microbial or anti-viral properties with increasing temperature, or a temperature sensor.

13. The facial garment of claim 1, wherein the textile mask body includes a conductive yarn arranged with insulative yarns to provide an electrostatic charge to provide antimicrobial or anti-viral properties.

14. The facial garment of claim 1, wherein the textile mask body includes at least one sensor configured to detect oximetry.

15. The facial garment of claim 14, wherein the at least one sensor includes a photoplethysmogram (PPG) sensor for sensing when oxygen level of the user is decreasing.

16. The facial garment of claim 1, wherein the textile mask body includes a form fitting yarn configured to heat shrink to provide form fit to the user's face.

17. The facial garment of claim 1, wherein the textile mask body includes shape memory yarn to provide a form fit to the user's face to reduce air gaps between the textile mask body and the user's face.

18. The facial garment of claim 1, wherein the textile mask body includes an infrared sensor positioned proximal to an ear of the user.

19. The facial garment of claim 1, wherein the textile mask body includes a photoplethysmogram (PPG) sensor configured to detect heart rate monitoring statistics.
